(19) United States
(12) Patent Application Publication
(10) Pub. No.: US 2025/0302913 A1
(43) Pub. Date: Oct. 2, 2025

(54) LACRIPEP PROMOTES NEUROREGENERATION AND MAINTAINS EPITHELIAL PROGENITOR CELL IDENTITY

(71) Applicant: The Regents of The University of California, Oakland, CA (US)

(72) Inventors: Sarah Knox, San Francisco, CA (US); Nancy McNamara, San Francisco, CA (US)

(73) Assignee: The Regents of the University of California, Oakland, CA (US)

(21) Appl. No.: 18/863,285

(22) PCT Filed: May 2, 2023
(86) PCT No.: PCT/US2023/020665

§ 371 Date: Nov. 5, 2024

Related U.S. Application Data
(60) Provisional application No. 63/339,075, filed on May 6, 2022.

(51) Int. Cl. A61K 38/16; A61K 9/00; A61P 27/02

(52) U.S. Cl. CPC A61K 38/16; A61K 9/0048; A61P 27/02

(57) ABSTRACT

Methods and compositions for stimulating nerve growth in the eye and skin and restoring damaged tissue are provided.

### CROSS-REFERENCE TO RELATED PATENT APPLICATIONS

The present application claims benefit of priority to U.S. Provisional Patent Application No. 63/339,075, filed May 6, 2022, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under grants R01 EY025980 and R01 EY026492 awarded by The National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Apr. 26, 2023, is entitled “081906-1326329-249600US_ST26.xml”, and is 8,352 bytes in size.

### BACKGROUND OF THE INVENTION

Tear deficiency due to lacrimal gland dysfunction (aqueous-deficient dry eye) is among the most common and debilitating outcomes of systemic autoimmune diseases including Sjogren's, rheumatoid arthritis, scleroderma and systemic lupus erythematosus (M. A. Lemp, C. Baudouin, J. Baum, M. Dogru, G. N. Foulks, S. Kinoshita, P. Laibson, J. McCulley, J. Murube, S. C. Pflugfelder, M. Rolando, I. Toda, in Ocular Surface (2007)). A healthy tear film provides an aqueous coating necessary for optimal vision and tissue function while also shielding the ocular surface from environmental, inflammatory, and microbial insult. Due to the essential requirement of tears in maintaining ocular health, corruption of tissue integrity and loss of homeostasis in response to prolonged dryness induce a vast array of pathological outcomes (S. C. Pflugfelder, C. S. de Paiva, Ophthalmology. 124, S4-S13 (2017)). Yet, despite the extensive ramifications of dry eye on ocular health and its significant impact on vision, quality of life, and the psychological/physical consequences of chronic pain (F. Stapleton, M. Alves, V. Y. Bunya, I. Jalbert, K. Lekhanont, F. Malet, K.-S. Na, D. Schaumberg, M. Uchino, J. Vehof, E. Viso, S. Vitale, L. Jones, Ocul. Surf. 15, 334-365 (2017)), there are currently only three clinically-approved therapies for the treatment of dry eye disease that specifically target T-cell mediated inflammatory pathways believed to be the primary driver of dry eye pathogenesis. None of these anti-inflammatory treatments are regenerative and promote modest improvements in the signs and symptoms of dry eye (S. C. Pflugfelder, C. S. de Paiva, Ophthalmology. 124, S4-S13 (2017)), which results in life-long corneal dysfunction and reduced quality of life.

### BRIEF SUMMARY OF THE INVENTION

In some embodiments, a method of regenerating functional sensory nerves in an eye of a human having damaged corneal nerves is provided. In some embodiments, the method comprises contacting the eye of the human with a polypeptide comprising KQFIENGSEFAQKLLKKFSLLKPWA (SEQ ID NO:1) in a dosage sufficient to regenerate functional sensory nerves in the eye.

In some embodiments, the human has an eye disorder selected from the group consisting of neurotrophic keratitis, Sjogrens, rheumatoid arthritis, Crohn's disease, radiation-damage (keratopathy), diabetic neuropathy, keratoconus, infectious keratitis, herpes simplex, herpes zoster, corneal dystrophies, atopic keratoconjunctivis, allergic conjunctivitis, glaucoma, Stevens-Johnson syndrome, toxic epidermal necrolysis, limbal stem cell deficiency, corneal pain, corneal neuralgia, penetrating keratoplasty, phototherapeutic keratectomy, chemotherapy-induced peripheral neuropathies, neuropathic dry eye and Parkinson's disease.

In some embodiments, the human has an eye disorder resulting from laser epithelial keratomileusis (LASEK), laser-assisted in situ keratomileusis (LASIK), photorefractive keratectomy (PRK), or small incision lenticule extraction (SMILE).

In some embodiments, the eye tissue has damaged corneal architecture and dosage is sufficient to improve corneal architecture.

In some embodiments, the polypeptide has an amino acid sequence that consists of SEQ ID NO:1. In some embodiments, the polypeptide consists of Ac-KQFIENGSEFAQKLLKKFSLLKPWA-NH2 (SEQ ID NO:5) or a salt thereof.

In some embodiments, the dosage is administered under a contact lens.

In some embodiments, the polypeptide comprises or is linked to a protein domain that adheres to the surface of the eye. In some embodiments, the protein domain binds to collagen, heparin, heparan sulfate, or a carbohydrate. In some embodiments, the protein domain is selected from the group consisting of a lectin carbohydrate-binding anchor domain, a von Willebrand factor (vWF) collagen-binding anchor domain, a Clostridium collagenase (ColH) collagen-binding anchor domain, and a heparin-binding (HS) anchor domain.

In some embodiments, a method of stimulating nerve regeneration in the skin or mouth in a human in need thereof is provided. In some embodiments, the method comprising contacting the skin with a polypeptide comprising KQFIENGSEFAQKLLKKFSLLKPWA (SEQ ID NO: 1) in an amount sufficient to stimulate nerve regeneration in the skin. In some embodiments, the human has a peripheral neuropathy resulting from a disorder selected from the group consisting of Systemic Lupus Erythematosus, diabetic neuropathy, radiation exposure, traumatic injuries or toxic agents, and wherein at least one symptom of the disorder is ameliorated.

In some embodiments, the polypeptide consists of SEQ ID NO:1. In some embodiments, the polypeptide has an amino acid sequence that consists of Ac-KQFIENGSEFAQKLLKKFSLLKPWA-NH2 (SEQ ID NO:5) or a salt thereof.

Also provide is a composition for ocular delivery comprising a polypeptide comprising KQFIENGSEFAQKLLKKFSLLKPWA (SEQ ID NO:1), wherein the polypeptide comprises or is linked to a protein domain that adheres to the surface of the eye. In some embodiments, the protein domain binds to collagen, heparin, heparan sulfate, or a carbohydrate. In some embodiments, the protein domain is selected from the group consisting of a lectin carbohydrate-binding anchor domain, a von Willebrand factor (vWF) collagen-binding anchor domain, a Clostridium collagenase (ColH) collagen-binding anchor domain, and a heparin-binding (HS) anchor domain.

### BRIEF DESCRIPTION OF THE DRAWINGS

FIG. 1A-K. Basal tear secretion, epithelial integrity and basal progenitor cell identity are restored with lacripep treatment during dry eye disease progression. A, Schematic of treatment regimen showing data collection time points for tissue analysis (top). B, Levels of physiological (basal) tear secretion at day 0, 7 and 15. C,D Lissamine green uptake (a measure of tissue penetration) in untreated/treated Aire KO corneas compared to age matched WT were assessed by scoring intensity of stain at day 7 and 15. The day 7 score was normalized to day 0 (before treatment) and the day 15 score was normalized to day 7. Data points above 1 indicate increased lissamine green uptake while points below 1 indicate reduced uptake. NUC=nuclei. E-H, Immunofluorescent analysis and quantification of the tight junction protein ZO1 (E,F), and basal progenitor cell marker KRT14 and differentiation marker KRT6A (G,H) at day 15. Arrows in G highlight basal cells co-expressing KRT6A and KRT14. The graph in H shows the percentage of basal KRT14+ basal cells co-expressing KRT6A. I, Quantification of proliferating basal epithelial cells at day 15. J-K, Immunofluorescent analysis and quantification of CD4+ T cells in WT and untreated/treated Aire KO corneas at day 15. Scale bars=50 μm. *p<0.05; **p<0.01; ***p<0.001; ****p<0.0001; ns=not significant; a one-way analysis of variance was applied to graphs B, D and F with the following post-hoc tests: a Tukey's multiple comparisons test was used in B, D and F, and a Dunett's T3 multiple comparisons test was used in Hand K. In graph I, a one-way analysis of variance with correction for multiple comparisons using a False Discovery rate of 0.05 and a two-stage step-up method of Benjamini, Krieger and Yekutieli. Each dot in the bar graphs represent a biological replicate. All data are expressed as mean+s.d. n>4 mice per group.

FIG. 2A-D. Sensory innervation of the desiccated cornea is restored in response to lacripep treatment. A, 3D projections of nerve fibers across the peripheral and central regions of WT and untreated, PBS- or lacripep-treated Aire KO cornea (whole mount). Nerves were immunolabeled for the remodeling neuronal marker GAP43, and sensory neurotransmitters substance P (SP), and calcitonin-gene-related peptide (CGRP). B, Quantification of GAP43+ nerves in WT, untreated, and treated Aire KO corneas. C, Central corneal cross-sections (10-12 μm) were immunolabeled for the pan-neuronal marker TUBB3 and nuclei (NUC). Scale bar=70 μm. D, Quantification of TUBB3+ nerves in the central corneal epithelium of WT, untreated and treated Aire KO. Scale bar=50 μm. *p<0.05; **p<0.01; ***p<0.001; a one-way analysis of variance was applied to data in graphs B and D (day 15). In graph D, a Student's T test was applied to WT vs KO at day 0. Each dot in the bar graph represents a biological replicate. All data are expressed as mean+s.d. n>4 mice per group.

FIG. 3A-E. Lacripep re-establishes functional corneal nerve-epithelial interactions during dry eye disease progression. A, Schematic of treatment regimen showing data collection time points for RNAseq and tissue analyses. B-C, Immunofluorescent analysis (B) and quantification (C) of regenerating nerves in the corneal epithelium at day 7. Graph in C shows the relative proportion of newly regenerating GAP43+ nerves in the central cornea. D, 3D reconstruction of whole mount immunofluorescent images of newly regenerating (GAP43+) and existing (TUBB3+) intraepithelial nerve terminals at day 7 (upper panel) and 15 (lower panel). E, Graph shows relative proportion of GAP43+, GAP43+ TUBB3+ and TUBB3+ nerve terminals in the central cornea at day 7. NUC=nuclei. Scale bar=25 μm, 10 μm. Each dot in the bar graphs represents a biological replicate. All data are expressed as mean+s.d. n>4 mice per group.

FIG. 4A-F. Lacripep activates master regulators of nerve regeneration in the dessicated cornea. A, RNA transcripts for syndecan-1 (Sdc1), co-receptor for lacripep, in the basal and suprabasal epithelial cells of WT and Aire KO cornea at 7 wks of age. Scale bar=50 μm. B, Volcano plot of differentially expressed corneal genes in response to lacripep versus PBS treatment at day 7. C, Gene Ontology (GO) analysis highlighting upregulated pathways in lacripep versus PBS-treated Aire KO corneas. D, Heatmap featuring lacripep-induced upregulated genes associated with axonogenesis, axon guidance, nerve-epithelial signaling and synapse formation at day 7 of treatment. E, Venn diagram of the top transcription factors Isl1, Rest and Rreb1 regulon targets upregulated in response to lacripep. NES>3, #Targets>100. F, GO analysis of the exclusive target gene sets for each TF in C, highlighting individual and combined roles in the regulation of function- and structure-based neuronal processes.

FIG. 5A-D. Lacripep improves tear secretion and barrier function despite chronic inflammation. A-B, Immunohistological analysis and quantification of CD4+ T cells in WT, untreated and treated Aire KO lacrimal glands. Graph in B shows the percentage of CD4+ T cells in each treatment group normalized to WT controls. C, Levels of pilocarpine induced (maximal) tear secretion at day 7 and 15 compared to baseline. D, RNAseq analysis of corneas at day 7 days of treatment highlights representative markers of various resident innate inflammatory cell populations. ****p<0.0001; a one-way analysis of variance was applied to B and C. Each dot in the bar graph represents a biological replicate. Error bars represent standard deviation. n≥4 mice per group.

FIG. 6A-C. RNAseq analysis of cornea samples at 7 days of treatment. A, Principal component analysis (PCA) plot of the different treatment groups. B, Gene Ontology (GO) analysis highlighting upregulated pathways in PBS- and lacripep-treated versus untreated Aire KO controls; p<0.05. C, Identification of top master transcriptional regulators enriched in lacripep-treated corneas when compared to PBS. iRegulon was employed on 886 upregulated genes. FC>1.5, p<0.05. Table show binding motif, Normalized enrichment score (NES) and Number of targets (#Targets). NES>3, #Targets>100.

### DEFINITIONS

Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which are described in Altschul et al., (1990) J. Mol. Biol. 215:403-410. Software for performing BLAST analyses is publicly available at the National Center for Biotechnology Information website, ncbi.nlm.nih.gov. The algorithm involves first identifying high scoring sequence pairs (HSPs) by identifying short words of length “W” in the query sequence, which either match or satisfy some positive-valued threshold score T when aligned with a word of the same length in a database sequence. T is referred to as the neighborhood word score threshold (Altschul et al., supra). These initial neighborhood words act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Regenerating functional sensory nerves” refers to restoring nerve function in the cornea (and optionally other epithelial organs). Regenerating functional sensory nerves can include (1) production of nerve-derived factors (e.g., Substance P, CGRP) that are released into the surrounding tissue to regulate organ function, homeostasis, and wound healing, and (2) establishment of nerve terminals that mediate nerve-cornea communication. Lacripep and other Lacritin peptides promote the return of a functional nerve supply through delivering these two outcomes. Functional innervation is indicated through restoration of physiological (basal) tear production as referenced below. Previously it was not understood that functional nerves sensing change at the ocular surface.

“Corneal architecture” refers to the multi-layered corneal epithelium (5-10 layers) that includes a basal layer of stem cell/progenitor cells that continuously give rise to the upper epithelial cell layers that play a role in barrier function such as the prevention of microbes and other materials from entering the ocular surface. During dry eye disease, barrier function is disrupted, caused in part by loss of cell-cell adhesions. Loss of cell-cell adhesions in dry eye disease is due to aberrant stem/progenitor cell differentiation to resupply the cell types required for barrier function. However, treatment with sufficient dosage of Lacritin peptides (e.g., lacripep) resolve this outcome via resupplying the corneal cells with nerves, thus resulting in the rescue of cell identity, cell differentiation and consequently, tissue architecture. Improvement in corneal architecture is demonstrated through restoration of cell-cell adhesion between neighboring superficial epithelial cells.

### DETAILED DESCRIPTION OF THE INVENTION

The inventors have determined that topical administration of synthetic peptides designed from Lacritin (e.g., lacripep™) regenerate multiple tissue compartments of the cornea and reactivate basal tear secretion, effectively returning the damaged, dysfunctional ocular surface to a near homeostatic state, and restoring physiological tear secretion. Appropriate peptide dosage resolves dry eye disease through reactivating basal tear secretion, restoring progenitor cell identity, rescuing epithelial barrier function, and re-establishing functional sensory innervation of the cornea. As shown below, Lacritin peptides (e.g., lacripep) achieve these outcomes without reducing ocular inflammation. However, notably, it does alter the composition of inflammatory cells, shifting from a pro-inflammatory to a pro-repair response, as described below.

Ocular inflammation is a significant mediator of dry eye. Indeed, dry eye predominately occurs in patients suffering autoimmune or chronic inflammatory diseases such as Sjogren's, rheumatoid arthritis, and lupus. Experimental dry eye models have shown that the inflammatory changes associated with dry eye have a role in its pathogenesis. First, adoptive transfer of CD4+ T cells from mice with dry eye to T-cell-deficient nude mice, leads to severe inflammation in the cornea, and conjunctiva, resulting in decreased tear production. Current anti-inflammatory therapies based on suppressing inflammation have shown little success in regenerating the cornea or effectively removing immune cells. Lacritin peptide (e.g., lacripep) treatment results in the acquisition of a pro-repair immune state, effectively limiting immune cell-mediated ocular damage. This provides a new therapeutic role for Lacritin peptides (e.g., lacripep) in disease management.

Sensory nerves derived from the ophthalmic lobe of the trigeminal ganglion primarily enter the corneal and establish nerve-epithelial interactions that serve an essential role in sensing changes at the ocular surface (e.g., dryness) and maintaining corneal epithelial homeostasis, in part, through activation of tear production from the lacrimal gland via the lacrimal reflex. Thus, the degree of basal tearing reflects the function and quality of sensory nerves within the corneal epithelium. Application of Lacritin peptides results in the re-establishment of sensory function, leading to the promotion of pro-secretory functions during dry eye disease progression.

In view of the above discoveries, it has been determined that one can regenerate functional sensory nerves in an eye of a human by applying a sufficient dosage of a polypeptide comprising SEQ ID NO: 1 to regenerate functional sensory nerves in the eye. This discovery has application to a number of ocular diseases that benefit from restoration of, for example corneal nerves, and regeneration of ocular epithelium. Moreover, in view of its effect of nerve re-growth, it is expected the polypeptides described herein will also find use in human skin disorders in which nerve growth is desired (e.g., skin disorders where epithelial neuropathy is experienced) and disorders affecting other mucosal membranes such as the mouth where damage to oral sensory nerves results in numerous clinical consequences (e.g., burning mouth syndrome, phantom oral sensations such as taste, touch and pain, as well as long term changes in food choice and body mass. See, e.g., Snyder et al., Rev Endocr Metab Disord. 2016 June; 17 (2): 149-158, describing mouth nerve disorders that can be ameliorated by contacting the mouth with the Lacritin peptides described herein.

Lacritin or various truncated active forms or synthetic analogs thereof can be used according to the methods described herein. Lacritin is an endogenous glycoprotein initially identified in tears (Sanghi, S. et al. J. Mol. Biol. 310, 127-139 (2001)). Lacritin's amino acid sequence is MKFTTLLFLAAVAGALVYAEDASSDSTGADPAQEAGTSKPNEEISGPAEPASPPETTTTA QETSAAAVQGTAKVTSSRQELNPLKSIVEKSILLTEQALAKAGKGMHGGVPGGKQFIEN GSEFAQKLLKKFSLLKPWA (SEQ ID NO:2). See, e.g., PCT Publication No. WO2015/138604. In addition, a variety of active fragments are known, including but not limited to those described in PCT Publication No. WO2015/138604 and US20190381136. In some embodiments, the active fragment comprises or consists of KQFIENGSEFAQKLLKKFSLLKPWA (SEQ ID NO:1). A synthetic version of this sequence, in which the amino terminus is acetylated and the carboxyl terminus is aminated (Ac-KQFIENGSEFAQKLLKKFSLLKPWA-NH2) (SEQ ID NO:5) can also be used. Commercial versions of this synthetic peptide are referred to as “Lacripep™” This group of peptides is referred to for convenience herein as “Lacritin peptides.”

A sufficient dosage of a Lacritin peptide to return a damaged, dysfunctional ocular surface to a homeostatic state will depend on the damage involved and the precise peptide and formulation used. In some embodiments, the dosage used is for a peptide as described above in a pharmaceutically-acceptable sterile solution, for example with three or more doses administered per day for a set number of days. In other embodiments, the peptide can be linked to a second protein domain or delivered via a contact lens, or both, to improve persistence of the peptide in the eye and enhance ocular surface uptake, thereby reducing the number of dosages per day and/or reducing the required concentration of the peptide delivered to the eye. The precise dosage of a formulation can be selected to achieve the desired endpoint, for example nerve regeneration or restoration of the surface of the eye.

In some embodiments, any of the above-described polypeptides is linked, optionally as a translational fusion protein, to a protein domain that adheres to the surface of the eye, i.e., has an affinity for the surface of the eye such that it is not readily washed away with saline solution or through the nature mechanisms of blinking, thereby raising the effect without an increased dosage due to retention of the active polypeptide on the eye. Exemplary protein domains that adhere to the eye are described in, e.g., WO2018057522. For example, exemplary protein domains that adhere to the eye and that can be linked to a Lacritin peptide can include but are not limited to collagen-binding polypeptides (e.g., von Willebrand factor (vWF) or Clostridium collagenase), a heparin-binding polypeptides (e.g., KRKKKGKGLGKKRDPSLRKYK (SEQ ID NO: 3) or KRKKKGKGLGKKRDPCLRKYK (SEQ ID NO: 4), or lectins (e.g., wheat germ agglutinin (WGA), concanavalin A (conA), and jacalin (Jac)). See, e.g., WO2018057522.

The above protein domains can fuse directly to a polypeptide comprising SEQ ID NO: 1 or SEQ ID NO:2 or via an amino acid linker, as a translational fusion protein. Accordingly, nucleic acids encoding such translational fusion proteins are also provided, as well as expression cassettes comprising a promoter operatively-linked to such nucleic acids and prokaryotic or eukaryotic cells comprising such nucleic acids, which can be used for example for production of the translational fusion proteins. Alternatively, the polypeptide comprising SEQ ID NO: 1 or SEQ ID NO:2 can be linked via chemical conjugation (i.e., not via a peptide bond) to the protein domain that adheres to the eye.

In addition, or alternatively, the polypeptide comprising SEQ ID NO:1 or SEQ ID NO: 2 can be otherwise delivered or formulated to sustain the polypeptide in the eye. Thus, in some embodiments, the polypeptide can be delivered using contact lens or to an eye wearing a contact lens such that the contact lens provides a sustained release of polypeptide and/or delays dilution or removal by the eye (e.g., via tearing) of the polypeptide. For example, in some embodiments, the Lacritin peptide can be delivered via the large reservoir under a scleral contact lenses so that the cornea is continuously bathed in a protected environment. Other devices for ocular delivery of drugs can also be used, such as those described in, e.g., US2013/0023838.

Sustained delivery of the Lacritin peptides can be achieved in a number of other ways as well. In some embodiments, the Lacritin peptide is delivered in a drug-eluting colloidal nanoparticle-laden contact lens that delivers the polypeptide at a steady rate over an extended period of time. Such delivery systems can comprise liposome encapsulation, microemulsions or micelles with high drug loading capacity to contain the Lacritin peptide. See, e.g., Choi et al., Materials (Basel). 2018 July; 11 (7): 1125 and Franco et al., Polymers 2021, 13, 1102. In some embodiments, the Laritin peptide is linked to vitamin E or d-α-Tocopheryl polyethylene glycol 1000 succinate to increase hydrophobicity and reduce the rate of drug release. See, e.g., Sharma et al., Journal of the Indian Chemical Society, Volume 99, Issue 3, March 2022, 100387 and Coruso, et al., Cornea 2016 February; 35 (2): 145-150.

The Lacritin peptides described herein can be formulated into a sterile solution adapted for delivery to the eye. The compositions can optionally contain other therapeutic agents that are suitable for treating or preventing a given disorder. Pharmaceutically carriers can enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

Pharmaceutical compositions as described herein can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions of the present invention. Applicable methods for formulating the polypeptides and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose of the polypeptides described herein is employed in the pharmaceutical compositions. The polypeptides can be formulated into pharmaceutically acceptable dosage forms by conventional methods known to those of skill in the art. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. It is especially advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

In some embodiments, the Lacritin peptide is delivered as eye drops. In some embodiments, the Lacritin peptide is delivered at a concentration of 0.4 to 40 micromolar. However, in some embodiments, the ability to sustain the presence of the Lacritin peptide over time plays at least as, or a more, significant role in reaching a desired endpoint than the precise concentration. Dosing can range for one, two, or three or more doses daily, for example when supplied in a non-sustained release formulation (e.g., eye drops). In some embodiments, the dosing schedule is at least three times daily depending on formulation, indication and disease severity, as three dosages per day has been found for eye drops to regenerate nerves. In some embodiment, the dosage exceeds twice daily at 4 micromolar, which has been found to be ineffective in achieving the end points described herein. In some embodiments in which the Lacritin peptide is delivered with a contact lens, disposable drug-eluting contact lenses (e.g., the Acuvue Theravision) can be worn for, e.g., 4-16 hours/day, e.g., up to 16 hours/day. In other embodiments providing for sustained release, for example but not limited to embodiments in which the Lacritin peptide is linked to a domain that anchors the peptide to the surface of the eye, fewer doses per day can cause the same end point. For example, a sustained release formulation or that otherwise resists removal of the peptide by tearing and/or blinking, can be delivered once, twice or three times a day.

Buffers can be used to adjust the pH to a desirable range for ophthalmic use. Generally, a pH of around 6-8 is desired, however, this may need to be adjusted due to considerations such as the stability or solubility of the therapeutically active agent or other excipients. In some embodiments, the buffer maintains the pH between 6.5 and 7.5. In other embodiments, the buffer maintains the pH between 7.0 and 7.4. Many buffers including salts of inorganic acids such as phosphate, borate, and sulfate are known. In some embodiments a phosphate/phosphoric acid buffer, e.g., a combination of phosphoric acid and one or more of the conjugate bases such that the pH is adjusted to the desired range, is used. In other embodiments a borate/boric acid buffer is used. In still other embodiments a citrate/citric acid buffer is used in the formulations described herein. In certain embodiments a combination of phosphate/phosphoric acid buffer and citrate/citric acid buffer is used in the formulations described herein.

In ophthalmically acceptable liquids, tonicity agents often are used to adjust the composition of the formulation to the desired isotonic range. Tonicity agents can include for example glycerin, mannitol, sorbitol, sodium chloride, and other electrolytes. In some embodiments of the invention, the tonicity agent is present in the formulation at a concentration of 1.20 to 1.25% w/v.

A surfactant may be used for assisting in dissolving an excipient or a therapeutically active agent, dispersing a solid or liquid in a composition, enhancing wetting, modifying drop size, or a number of other purposes. Useful surfactants, include, but are not limited to sorbitan esters, Polysorbate 20, Polysorbate 40, Polysorbate 60, Polysorbate 80, stearates, glyceryl stearate, isopropyl stearate, polyoxyl stearate, propylene glycol stearate, sucrose stearate, polyethylene glycol, polyethylene oxide, polypropylene oxide, polyethylene oxide-polypropylene oxide copolymers, alcohol ethoxylates, alkylphenol ethoxylates, alkyl glycosides, alkyl polyglycosides, fatty alcohols, phosphalipids, phosphatidyl chloline, phosphatidyl serine, and the like.

Other excipient components which may be included in the ophthalmic preparations are chelating agents. A useful chelating agent is edetate disodium, although other chelating agents may also be used in place or in conjunction with it.

Preservatives are used in multi-use ophthalmic compositions to prevent microbial contamination of the composition after the packaging has been opened. A number of preservatives have been developed including quaternary ammonium salts such as benzalkonium chloride; mercury compounds such as phenylmercuric acetate and thimerosal; alcohols such as chlorobutanol and benzyl alcohol; and others.

In part in view of the discovery that Lacritin peptides regenerate multiple tissue compartments of the cornea and reactivate basal tear secretion, effectively returning the damaged, dysfunctional ocular surface to a near homeostatic state, it has been determined that a number of ocular disorders can be treated than previously realized. Moreover, additional therapeutic effects can be imparted in a number of known eye disorders, which effects can be achieved upon sufficient dosage.

In view of the discoveries described herein, a number of ocular diseases or disorders can be treated or ameliorated by administration of a sufficient dosage of a Lacritin peptide as described herein. For example, in some embodiments, a Lacritin peptide as described herein is administered to an eye of a human having an eye disorder selected from the group consisting of neurotrophic keratitis, Sjogrens, rheumatoid arthritis, Crohn's disease, radiation-damage (keratopathy), diabetic neuropathy, keratoconus, infectious keratitis, herpes simplex, herpes zoster, corneal dystrophies, atopic keratoconjunctivis, allergic conjunctivitis, glaucoma, Stevens-Johnson syndrome, toxic epidermal necrolysis, limbal stem cell deficiency, corneal pain, corneal neuralgia, penetrating keratoplasty, phototherapeutic keratectomy, chemotherapy-induced peripheral neuropathies, neuropathic dry eye and Parkinson's disease. Each of these disorders will be ameliorated by stimulating corneal nerve growth to normal levels. For example, by restoring nerves in the eye, corneal pain can be alleviated. In some embodiments, the human has received one or more of laser epithelial keratomileusis (LASEK), laser-assisted in situ keratomileusis (LASIK), photorefractive keratectomy (PRK), or small incision lenticule extraction (SMILE).

In yet other embodiments, a therapeutic dose of one of more Lacritin peptide is contacted to skin in a human in need thereof, thereby improving or initiating innervation. This aspect can be used, for example, in humans experiencing peripheral neuropathy. For example, in some embodiments, a human receiving a Lacritin peptide on the skin can include, but are not limited to, humans experiencing systemic lupus erythematosus, diabetic neuropathy, radiation exposure, traumatic injuries or exposure to toxic agents.

Accordingly, Lacritin peptides as described herein can be formulated for delivery to the skin. The carrier may be any gel, ointment, lotion, emulsion, cream, foam, mousse, liquid, spray, suspension, dispersion or aerosol which is capable of delivering active ingredients to and into skin. A penetration enhancer may be added to enable the active agents to cross the barrier of the stratum corneum. In some embodiments, the Lacritin peptides are formulated into a transdermal system, for example but not limited to a bandage or patch, for extended release of the active ingredient into the skin. See, e.g., US Patent Publication No. 2009/0062394.

In some embodiments, the transdermal carrier comprises an adhesive. Suitable adhesives are known in the art and include pressure-sensitive adhesives and bioadhesives. Bioadhesive materials useful in some embodiments include those described in U.S. Pat. No. 6,562,363. For example, bioadhesive materials may include polymers, either water soluble or water insoluble, with or without crosslinking agents, which are bioadhesive. Exemplary bioadhesives include natural materials, cellulose materials, synthetic and semi-synthetic polymers, and generally, any physiologically acceptable polymer showing bioadhesive properties, or mixtures of any two or more thereof.

### Example

Due to the fundamental requirement for tears in corneal maintenance and the clear role of desiccating stress as a principal driver of dry eye pathogenesis, there has been a recent focus on the application of tear-promoting factors to relieve dry eye. Lacritin, an endogenous glycoprotein identified in tears that is deficient in dry eye patients (4), has been found to possess pro-secretory properties in healthy and diseased animal models (4, 5), and to promote corneal epithelial cell proliferation in vitro (6). These findings led to the development of lacripep, a stable synthetic peptide consisting of lacritin's active C-terminal fragment, that has also been shown to stabilize the human tear film (7). However, its impact on cornea regeneration, including tissue architecture and epithelial cell identity, integrity and homeostasis, as well as physiological tear secretion, during dry eye disease progression has not been investigated. Furthermore, whether application of lacripep to the desiccated cornea can restore the significantly depleted functional nerve supply that is essential for basal tear secretion, ocular surface integrity, and corneal wound healing/tissue regeneration, remains unknown.

Here, using the well-characterized, autoimmune regulator (Aire)-deficient mouse model of spontaneous, autoimmune-mediated dry eye, we show that topical administration of lacripep resolves dry eye disease through reactivating basal tear secretion, restoring progenitor cell identity, and rescuing epithelial barrier function. Lacripep achieves these outcomes through re-establishing functional sensory innervation of the corneal epithelium, and can do so despite the presence of chronic ocular inflammation. Thus, we have identified the first regenerative ocular therapeutic for dry eye disease that restores the secretory and epithelial integrity of the desiccated cornea through promoting sensory reinnervation.

### Results

Basal Tear Secretion, Epithelial Integrity and Basal Progenitor Cell Identity are Restored with Lacripep Treatment During Dry Eye Disease Progression

The process of reflex (basal) tear secretion that occurs in response to changes at the ocular surface is essential to corneal function, homeostasis and wound healing and is profoundly reduced in human patients with dry eye disease. Although lacritin has been shown to promote basal tearing in a healthy rabbit model (5), whether lacripep is also capable of increasing basal tear production and can do so during dry eye disease progression is unknown. To test this, we evaluated basal tearing by utilizing the Aire mouse model of aqueous deficient dry eye (8). Aire knockout (KO) female mice develop classic signs of dry eye disease over a 2-week time period. This begins with small lymphocytic infiltrates in the lacrimal glands and mildly reduced tear secretion, corneal innervation, and barrier function at 5 weeks (wks), consistent with mild aqueous-deficient dry eye, that progresses to extensive CD4+ T cell-mediated exocrinopathy, severe tear reduction, and corneal pathologies at the epithelial, stromal and nerve-associated levels by 7 wks (8-10).

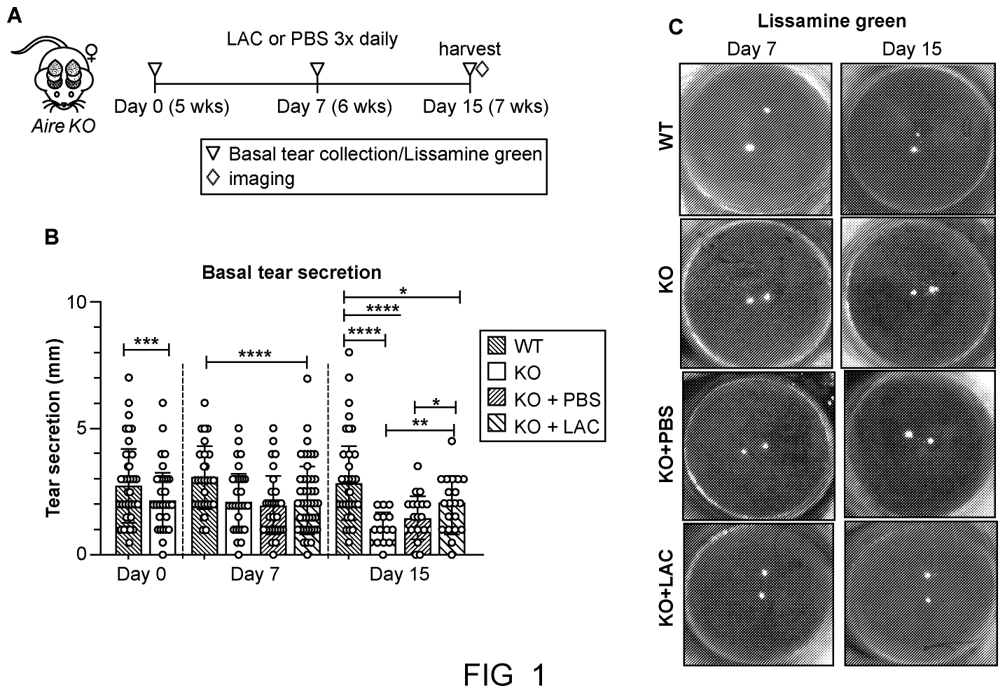

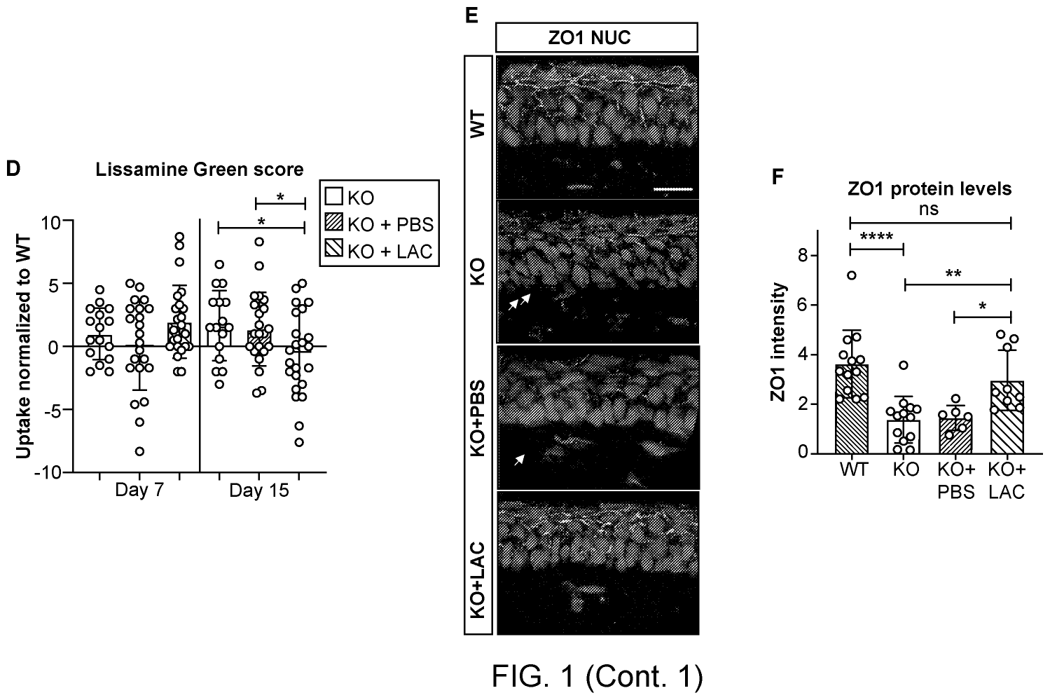

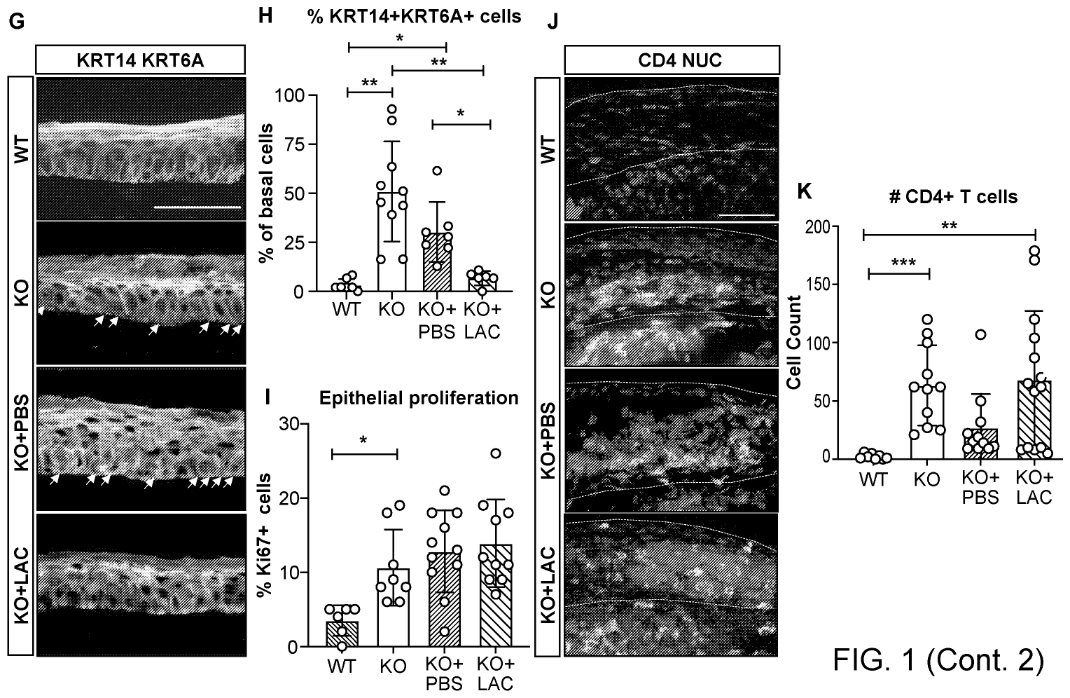

To determine the impact of lacripep on physiological (basal) tear secretion, lacripep (4 μM) or phosphate buffered saline (PBS, control) was topically applied to Aire KO corneas 3 times (3×) per day for 14 consecutive days (from 5 to 7 wk of age) and tear production was measured at day 0, 7 and 15 (FIG. 1A). Basal tear levels for the untreated Aire KO mice were significantly reduced at day 0 (77% of WT levels) and continued to decrease over time (67% at day 7 and 37% at day 15) (FIG. 1B). Tearing was also significantly decreased for the PBS-treated mice, with tear volumes being 50% of the WT controls at day 15 (FIG. 1B). Although lacripep-treated Aire KO mice showed similar levels of tear production as untreated and PBS-treated mice at day 7, at day 15 tear volumes remained at day 0 levels (83% of WT) (FIG. 1B), demonstrating lacripep retains pro-secretory functions during dry eye disease progression.

As dry eye results in disruption of ocular surface integrity resulting in cornea barrier dysfunction and, consequently, fluid loss and pathogen invasion (11), we next determined whether lacripep maintains corneal epithelial architecture by analyzing lissamine green uptake at day 7 and 15. At day 7 all Aire KO treatment groups exhibited increased lissamine green uptake compared to WT controls (FIG. 1C,D). However, at day 15, epithelial barrier function in lacripep- but not PBS-treated or untreated corneas was significantly improved, with uptake levels resembling those of WT corneas (FIG. 1C,D). Restoration of barrier function was further confirmed by analyzing the superficial epithelial layers for the tight junction protein ZO1 which is essential to the structural integrity of the corneal epithelium (12). Levels of ZO1 were greatly reduced in the untreated and PBS treated Aire KO cornea (FIG. 1E,F). In contrast, ZO1 expression in lacripep-treated corneas mimicked that of WT controls (FIG. 1E,F), thus, demonstrating lacripep is sufficient to rescue functional integrity of the corneal epithelium.

Although it has been well established that basal progenitor cells (also referred to as transit amplifying cells (13)) undergo mitosis and migration (14) to give rise to the barrier forming suprabasal epithelial and stratified squamous epithelial cells, the impact of dry eye disease on basal cell identity and differentiation remains largely unknown. Thus, we questioned whether basal cell identity was altered in the desiccated cornea through defining the cellular location of KRT6A, a marker of early epithelial cell differentiation in skin (15) and cultured cornea epithelial cells (16). Accordingly, we confirmed that KRT6A marks differentiated corneal epithelial cell types in vivo, as shown by its exclusive expression in KRT14-deficient suprabasal and superficial epithelial cells of healthy cornea at 7 wks of age (FIG. 1G). In contrast, KRT6A appeared in a large cohort of KRT14+ basal cells in the untreated Aire KO cornea (FIG. 1G, arrows), suggesting progenitors were undergoing aberrant differentiation. Treatment of Aire KO cornea with PBS for 2 wks did not reverse this outcome, with the number of KRT6A+ KRT14+ cells being similar to untreated controls (FIG. 1G, H). Strikingly, however, cell identity in lacripep-treated corneas returned to that of WT tissue, with KRT6A remaining exclusively expressed by suprabasal/superficial cells (FIG. 1G, H). To determine if changes in cell identity alters progenitor cell division, we analyzed the percentage of proliferating K14+ basal cells in treated and untreated corneas. In the untreated Aire KO corneas, K14+ cell division was significantly increased, as denoted by Ki67, compared to WT controls (FIG. 1I). A similar outcome was found for both PBS- and lacripep-treated Aire KO corneas, indicating cell identity but not cell division is impacted by lacripep.

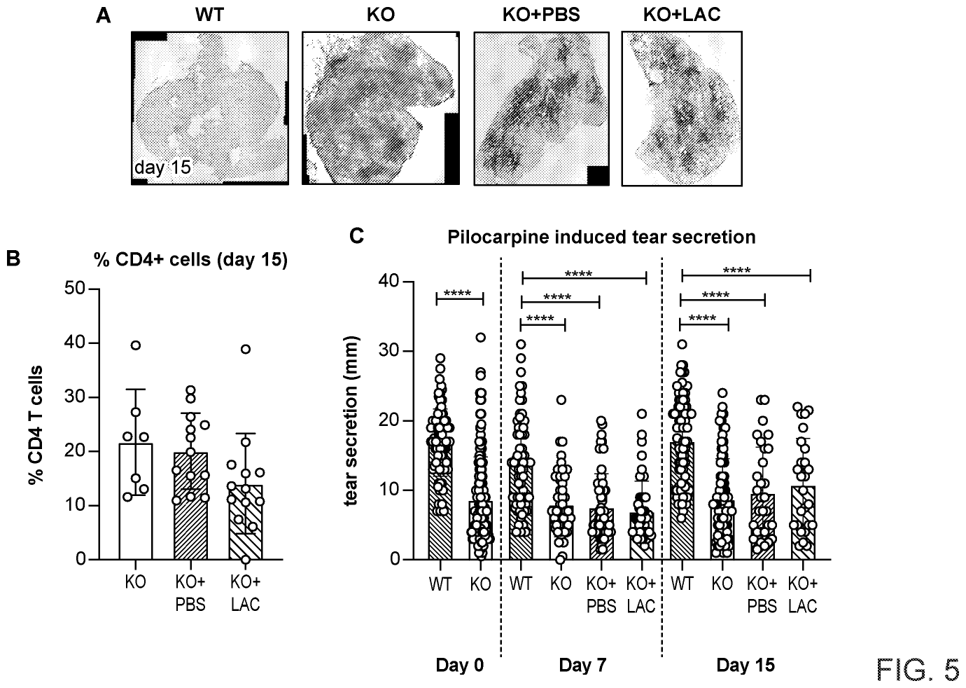

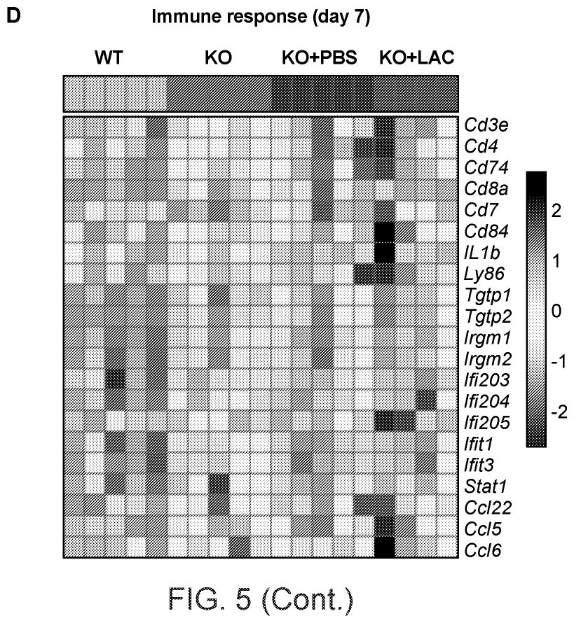

We next tested whether lacripep restored basal cell identity and barrier function by reducing immune cell infiltration. Prolonged desiccating stress from reduced tear production elicits an immune response in which activated CD4+ T cells home to the corneal epithelium and stroma (primarily accumulating at the limbal region) to modulate epithelial differentiation (17) and direct the development of autoimmune mediated dry eye (18). Intriguingly, at day 15 we found all Aire KO corneas (FIG. 1I,J), as well as their corresponding lacrimal glands (FIG. 5A, B), regardless of treatment, to be extensively infiltrated by CD4+ T cells. Furthermore, the extent of immune mediated damage to Aire KO gland function under each condition was comparable as there was no difference between maximal tear secretion in response to systemic injection of the muscarinic agonist pilocarpine (9,19). This outcome was further supported by RNAseq analysis at day 7 that showed (i) the expression of T cell markers such as Cd3, Cd7, and Cd8, (ii) T cell specific response genes, such as T cell-specific guanine nucleotide triphosphate-binding protein 1 and 2 (Tgtp1 and Tgtp2), and (iii) macrophage markers, such as Cd64, remained significantly elevated in the untreated and PBS/lacripep treated Aire KO corneas compared to the WT controls (FIG. 5D). In addition, key inflammatory mediators that are typically induced in desiccated corneas, including interleukin-1 beta (IL1b), and interferon response genes (Irgm1, Ifi203, Ifit1, Ccl22, Ccl5, and Stat1) were expressed at similar levels in all diseased corneas (FIG. 5D).

Thus, together these data indicate that, despite the ongoing, large-scale inflammatory response that occurs during dry eye disease progression, lacripep is capable of improving corneal barrier function and rescuing cell identity in the desiccated cornea without dampening inflammation.

### Innervation of the Desiccated Cornea is Restored in Response to Lacripep Treatment

As basal tear production is dependent on sensory innervation of the cornea, and dry eye leads to diminished corneal innervation in humans (20, 21) and mice (8, 22), we next determined whether the maintenance of reflex tearing in response to lacripep was due to restoration of the nerve supply. Sensory nerves derived from the ophthalmic lobe of the trigeminal ganglion primarily enter the corneal stroma at the limbal region and extend along the basement membrane, with axon fibers branching upward through the multi-layered epithelium to establish nerve-epithelial interactions. Here, they serve an essential role in sensing changes at the ocular surface (e.g., dryness) and maintaining corneal epithelial homeostasis, in part, through activation of tear production from the lacrimal gland via the lacrimal reflex. Thus, the degree of basal tearing reflects the function and quality of sensory nerves within the corneal epithelium. Consistent with this requirement, basal tear production and corneal nerve fiber density are significantly reduced in the Aire KO mouse model (beginning at 5 wks (9)) similar to human patients suffering from dry eye disease e.g., due to Sjögren's (21, 23), an outcome that strongly correlates with loss of active lacritin in human tears (24).

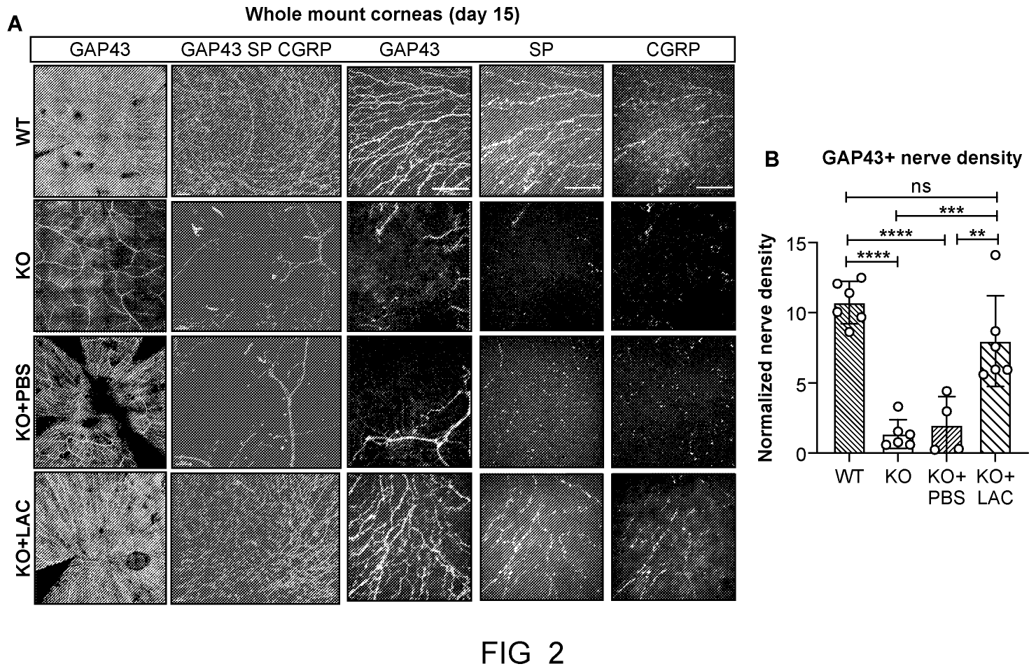

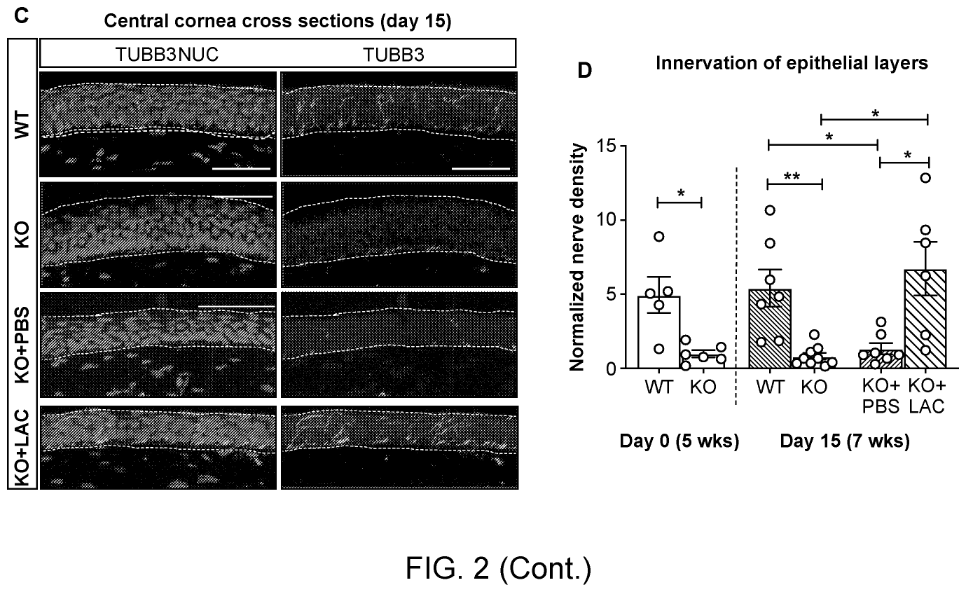

To test for changes in innervation, corneas isolated from the four treatment groups at day 15 were immunostained for growth-associated protein 43 (GAP43), a marker of remodeling axons (25), along with two common sensory neuropeptides, substance P (SP) and calcitonin-gene related protein (CGRP). SP and CGRP are differentially expressed to carry out discrete functions: SP is released in response to trigeminal activation to modulate tear secretion and goblet cell function (26) while CGRP is involved in multiple homeostatic processes, including corneal epithelium regeneration and regulation of vasculature (27, 28). As shown in the whole mount images in FIG. 2A, WT corneas showed extensive innervation by GAP43+ nerve fibers expressing SP and/or CGRP (FIG. 2A,B), consistent with the constant remodeling and functionality of corneal nerves (24). In contrast to WT, nerve density was dramatically reduced in the untreated and PBS-treated Aire KO corneas, indicating lubrication alone is not sufficient to maintain a functional nerve supply (FIG. 2A). Strikingly, however, lacripep-treated corneas showed extensive innervation throughout the tissue, with the density of highly branched GAP43+ nerve fibers expressing SP and CGRP being nearly equivalent to that of the WT controls (FIG. 2A,B). Thus, lacripep treatment successfully regenerates the sensory nerve supply to the inflamed cornea during disease progression.

### Lacripep Re-Establishes Functional Corneal Nerve-Epithelial Interactions During Dry Eye Disease Progression

Given nerve-epithelial interactions are essential for mediating physiological tear secretion, we next assessed innervation to corneal epithelial cell layers at 2 wks of treatment (day 15, 7 wks of age) through immunofluorescent analysis. As shown in FIG. 2C, the corneal epithelium of untreated and PBS-treated mice were highly deficient in axons compared to the extensively innervated WT controls (FIG. 2C,D). In contrast, innervation of lacripep-treated epithelial cell layers resembled that of WT tissue, with TUBB3+ axons extending apically through the epithelial layers to reach the most superficial cells (FIG. 2C,D). Thus, the reduced epithelial innervation at 5 wks is effectively restored through sustained lacripep treatment.

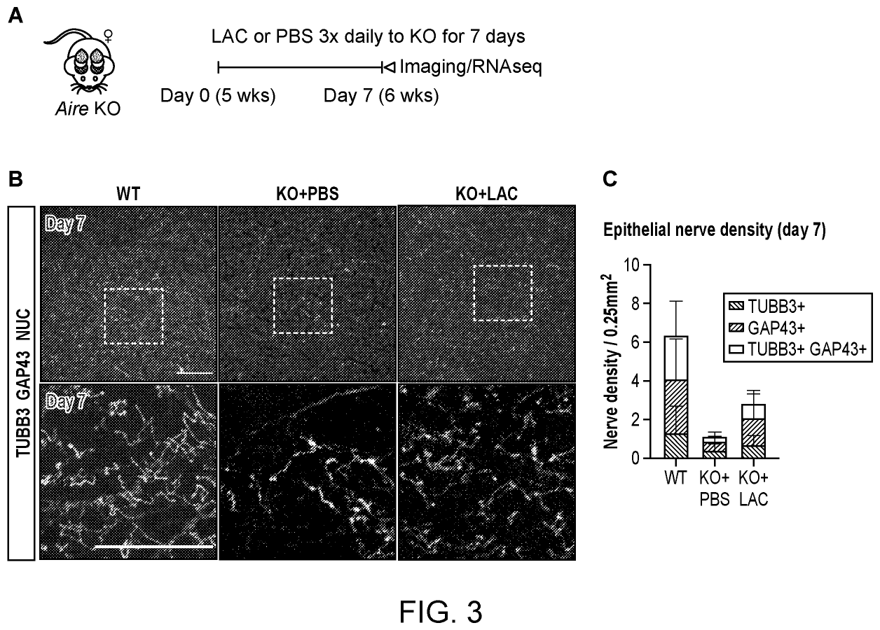

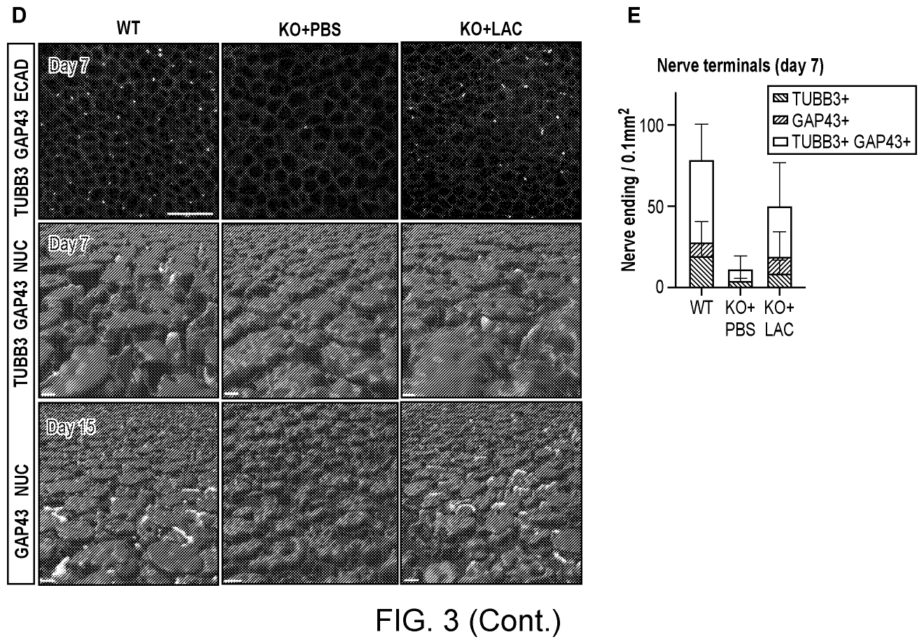

Next, we questioned the timing of epithelial reinnervation in response to lacripep treatment by analyzing the nerve supply at the 7 day time point (6 wks of age) through 3D imaging of intact whole mount corneas (FIG. 3A). As shown in FIG. 3B, PBS-treated Aire KO corneal epithelia showed a severe reduction in innervation at day 7, with the central cornea possessing only 18% of the nerve density of WT tissue (FIG. 3C), an outcome that remained at this level over the 2 wk treatment window (see FIG. 2). In contrast, corneas treated with lacripep exhibited significantly greater innervation at 7 days, reaching 45% of the nerve density of WT tissue (FIG. 3B,C), with levels returning to that of WT cornea by day 15 (see FIG. 2). Consistent with lacripep promoting nerve regeneration rather than nerve maintenance, lacripep-treated Aire KO corneas displayed a greater proportion of newly regenerating GAP43+ (TUBB3−) nerves relative to GAP43+ TUBB3+ nerves than the WT corneas (FIG. 3B,C). Specifically, the central corneas of lacripep-treated KO mice were populated with 50% GAP43+ nerves and 26% GAP43+ TUBB3+ nerves while the WT tissue showed 43% and 37%, respectively (FIG. 3C). Of note, the pro-regenerative cellular changes after axon injury result in neurons switching from an active, electrically transmitting state back to an electrically silent, growth-competent state (29-31), thereby likely impairing increased basal tear production at the 7 day time point for lacripep treated mice compared to untreated Aire KO controls (FIG. 1B).

We next determined the extent of functional reinnervation of the cornea at day 7 by assessing the distribution of sensory nerve endings (nerve terminals) within the epithelial layers in lacripep-treated Aire KO corneas compared to WT and PBS KO controls (FIG. 3D). Sensory nerve terminals within the corneal epithelium respond to changes in the thickness and stability of the tear film to promote basal tear secretion, while also triggering epithelial responses to restore homeostasis through the release of neurotransmitters. Using 3D imaging and topographical reconstruction by IMARIS, we identified an extensive array of intraepithelial sensory nerve terminals within lacripep-treated epithelia (50 per 0.1 mm²) compared to the PBS-treated (5 per 0.1 mm²) and WT controls (78 per 0.1 mm²) (FIG. 3D,E). Furthermore, consistent with nerve regeneration rather than nerve maintenance, we observed an increase in the proportion of the sensory nerve terminals that expressed GAP43 as opposed to only TUBB3 in the lacripep-treated corneas when compared to WT controls (FIG. 3D,E). Moreover, at day 15 the total number of nerve terminals in the lacripep treated Aire KO cornea was similar to that of WT controls highlighting the extensive recovery of innervation taking place during disease progression (FIG. 3D). Collectively, our data illustrate the therapeutic efficiency of lacripep as the first topical dry eye treatment capable of regenerating functional corneal nerves and nerve-epithelial connections required for ocular integrity, signaling, and tear secretion.

### Lacripep Activates Master Regulators of Nerve Regeneration

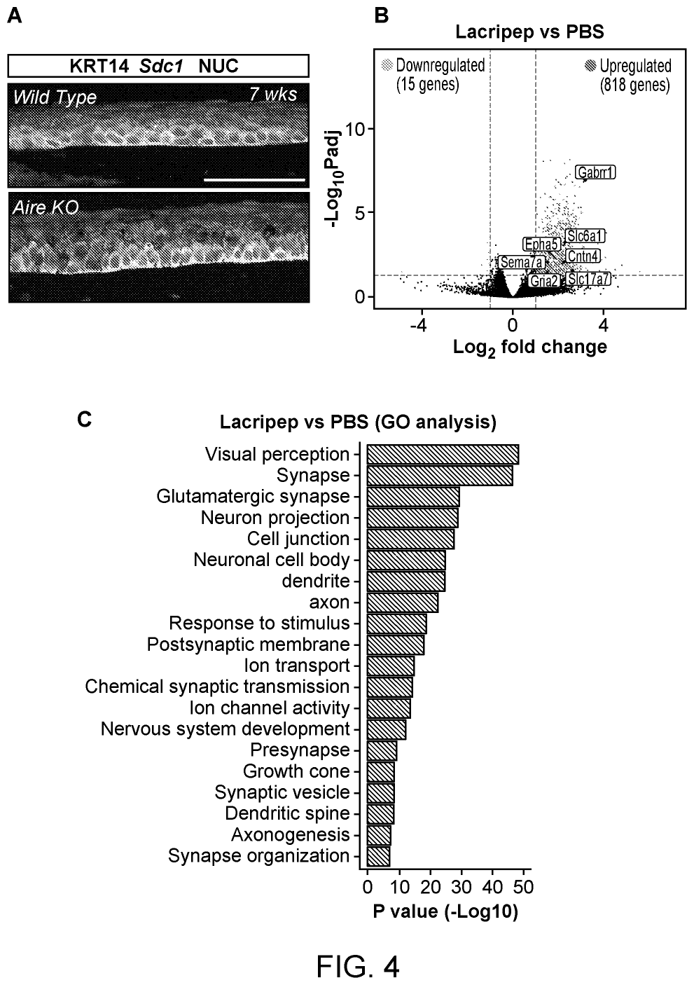

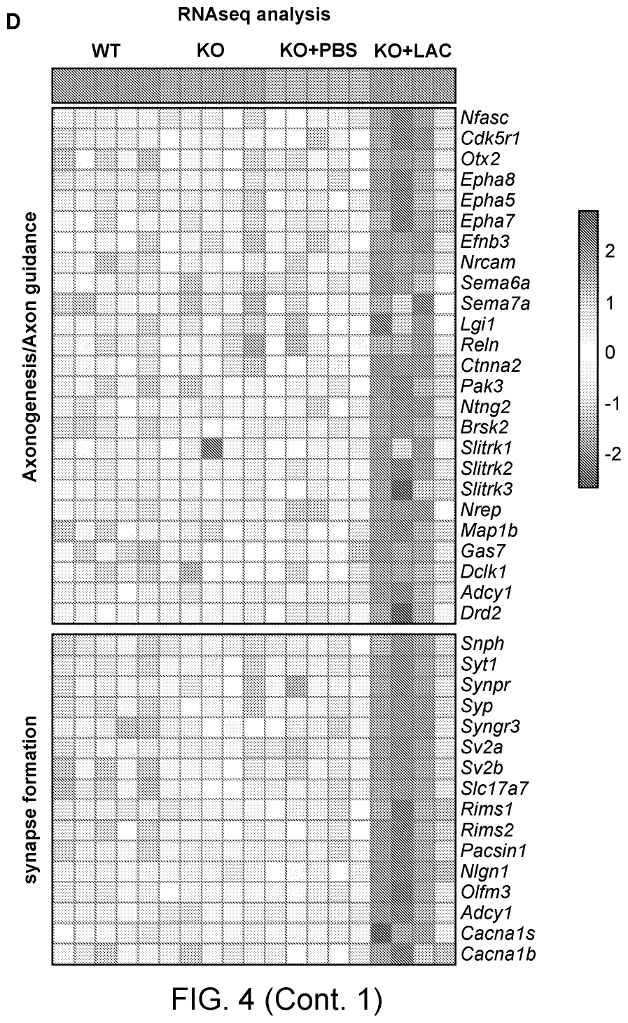

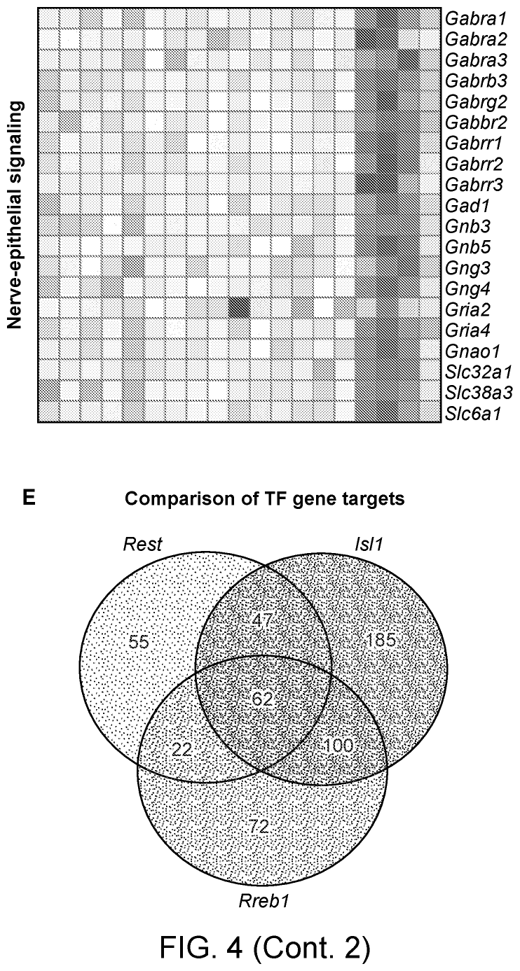

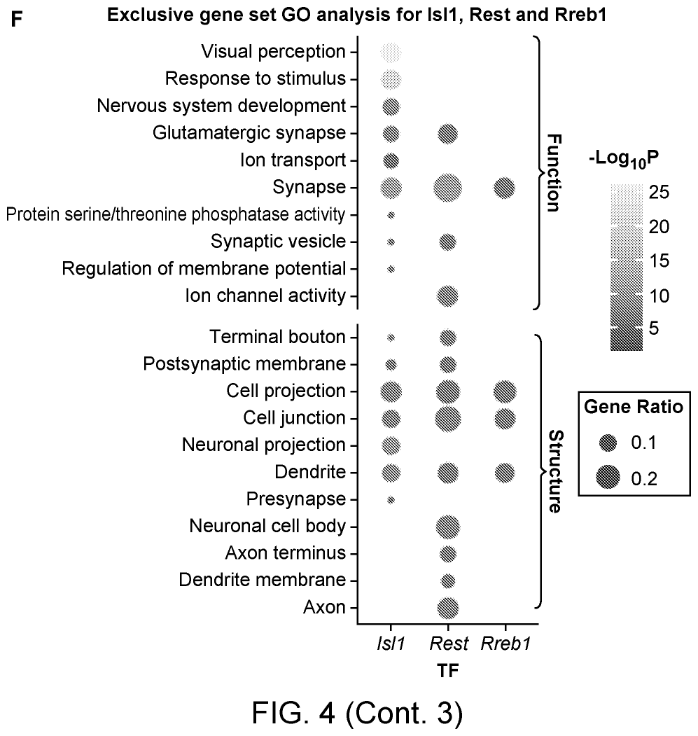

Finally, we questioned the potential mechanism by which lacripep restores the nerve supply. Lacritin and lacripep bind and activate syndecan-1 (SDC1), a transmembrane heparan sulfate proteoglycan enriched in the corneal epithelium, which has been shown to increase epithelial cell proliferation and migration in vitro (32-33). SDC1 is also heavily involved in corneal nerve regeneration after injury (34), with Sdc1-deficient mice exhibiting a profound reduction in corneal innervation after ocular damage that is subsequently followed by corneal pathologies consistent with dry eye (34). Thus, we first confirmed that SDC1 remains expressed in the epithelium during dry eye disease progression through application of in-situ hybridization (RNAscope) to healthy and diseased corneas at 7 wks of age. As shown in FIG. 4A, Sdc1 transcripts were highly enriched in the basal and suprabasal cell populations of the WT corneas in a manner consistent with previous studies (35). Similarly, we found Sdc1 transcripts were also abundantly located in the basal and suprabasal cells of the Aire KO corneal epithelium (FIG. 4A), thus demonstrating that SDC1-lacripep interactions within the epithelium can take place during dry eye disease progression.

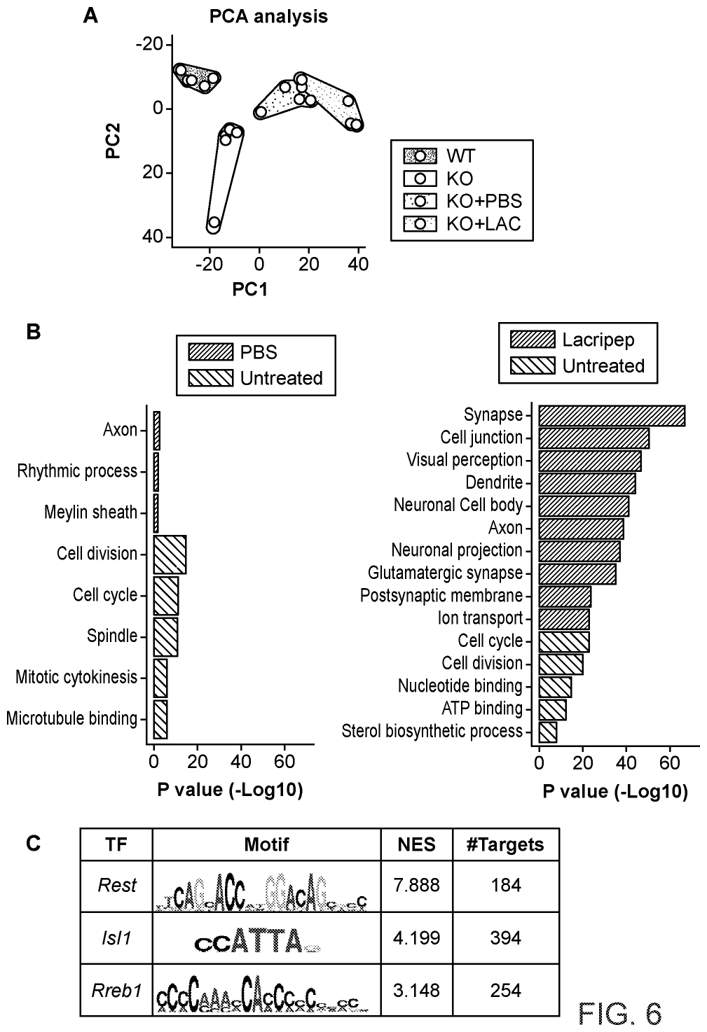

To identify potential downstream targets of lacripep treatment, we elucidated changes in corneal gene expression and signaling pathways at the day 7 time point through RNAseq. Principal component analysis (PCA) identified major transcriptional differences between each treatment group (FIG. 6A). Strikingly, differential gene expression analysis (DESeq2 (36); Padj<0.05; 2-fold change cutoff) of lacripep-versus PBS-treated corneas revealed an extensive alteration in gene expression that was predominantly associated with increased transcription, with 818 genes being significantly upregulated while only 15 were downregulated (FIG. 4B). Subsequent analysis yielded enriched gene sets that were highly associated with neuronal gene ontology (GO) terms e.g., synapse (P=3.6E-47), visual perception (P=5.8E-49), neuron projection (P=2.1E-29), and response to stimulus (P=1.8E-19) (FIG. 4C). These included cohorts of genes expressed by nerves and/or corneal epithelial cells involved in axonogenesis and axon guidance e.g., Neurod2, Cntn4, ephrins (Epha5, Epha7, Epha8) and semaphorins (Sema6a, Sema7a); synapse formation and function e.g., synaptic vesicle membrane proteins (Syt1; synaptophysin-Syp) and synaptic vesicle exocytosis regulators (Rims1, Rims2); neuronal signaling e.g., GABA receptors (Gabra2, Gabrr1), glutamate receptors (Gria2, Gria4), and G protein signal-transducing mediators (Gnb3, Gng4); and neurotransmitter transport e.g. glutamate transporters (Slc17a7) and GABA transporters (Slc6a1, Slc32a1) (FIG. 4D). Analysis of corneas treated with lacripep or PBS versus untreated corneas further emphasized the ability of lacripep and not PBS alone to induce transcription of neuronal gene sets (FIG. 6B). Combined, these data clearly demonstrate that lacripep positively promotes neuroregeneration rather than to preserving corneal innervation by upregulating a gene signature associated with axon migration, synapse formation and neuronal-epithelial signaling.

To identify candidate transcription factors that regulate these gene sets, we performed transcription factor (TF)-gene target regulatory network analysis using iRegulon (37). iRegulon identifies transcription factor-binding motifs that are enriched in the genomic regions of a query gene set and predicts transcription factors that bind to them. This revealed an abundant upregulation of target gene sets from 3 top master transcriptional regulators, RE1 Silencing Transcription Factor (Rest, 184 genes), ISL LIM Homeobox 1 (Isl1, 394 genes), and Ras-Responsive Element-Binding Protein 1 (Rreb1, 254 genes) (FIG. 6C, FIG. 4E). ISL1 plays an essential role in the generation of sensory and sympathetic neurons (38 39), REST is a master regulator of neurogenesis that plays a role in modulating synaptic plasticity (40-42) and RREB1 regulates axon injury (43). Notably, we identified a significant number of genes per gene set that are uniquely regulated by 1 of the 3 different TFs (FIG. 4E). This was particularly the case for Isl1, a TF previously shown to be enriched in the limbal cells (44) and corneal sensory nerves (45), where 47% of gene targets (185 out of 394) did not overlap with the other TFs, compared to 29% and 28% for Rest and Rreb1, respectively (FIG. 4E). The Isl1 exclusive gene set was also extensively enriched in genes involved with functional and structural nerve-mediated processes including visual perception (P=3.5E-27, e.g., Capb4, Vsx1), response to stimulus (P=9.7E-19; e.g., Slc17a7, Ush2a), synapse (P=9.4E-8, e.g. Magi2, Synpr) and neuron projection (P=6.4E-6, e.g., Cdh23, Kif1a) (FIG. 4F). Although the identified gene sets found to be exclusively regulated by Rest and Rreb1 were less extensive than that of Isl1, each of the 3 TFs regulated specific genes involved in synapse formation and function (FIG. 4F), suggesting that Isl1, Rest and Rreb1 jointly regulate nerve-epithelial communication in response to lacripep.

Thus, together these data suggest that lacripep regenerates sensory nerves and re-establishes nerve-epithelial communication during dry eye disease progression through upregulation of master regulators of neuronal-associated gene sets.

### Discussion

To date, none of the dry eye therapies, including those currently in clinical trials, have been shown to promote the comprehensive restoration of corneal structure and function at the cellular and tissue level in pre-clinical animal models. Indeed, current medications for multiple ocular surface diseases serve to inhibit inflammation but fail to act on the tissue itself to retain or regain structure and function. Here, we demonstrate that lacripep treatment reverses the multifaceted pathogenesis of dry eye disease through its prosecretory, pro-regenerative, and neurotrophic functions. Using a murine model that recapitulates many of the features of dry eye disease, we show that lacripep restores the structural and functional integrity of the cornea and basal progenitor cell identity by promoting functional reinnervation of the epithelium, thereby serving to disrupt disease development and support active wound healing. Furthermore, we show that this outcome occurs in the presence of chronic inflammation, thus indicating that the regeneration and reinnervation of inflamed tissues can occur in the diseased context.

Loss of corneal nerves is an established clinical consequence of dry eye pathogenesis in Sjögrens, diabetes (46), rheumatoid arthritis (47), scleroderma (48), thyroid associated disorders (e.g., thyroid-associated ophthalmopathy) (49) and chronic graft-versus-host disease (50). However, despite this, the clinically approved therapeutics utilized to treat dry eye to date are directed at dampening inflammation but none have been shown to promote the restoration of corneal nerves or to regenerate tissue. With recent studies showing a vital role for T-cell signaling pathways in promoting the stability of ocular surface homeostasis and the wound repair process (51, 52), reducing immune cell infiltration and/or altering their function may inhibit cornea/nerve regeneration. In contrast, the highly utilized anti-inflammatory drug cyclosporin A has been shown to retard the regeneration of surgically transected nerves (53). These outcomes have consequently fueled a growing interest in the development of novel therapies that target disrupted innervation. To date, several factors with neuroregenerative potential have been tested in animal models, including corneal matrix repair product cacicol (54), insulin growth factor-1 (55), the neuropeptide pituitary adenylate cyclase-activating polypeptide (56), pigment-epithelium derived factor (57), and N-(1-acetylpiperidin-4-yl)-4-fluorobenzamide (FK962) (58) but results have been limited. More recently in preclinical and clinical studies for treating neurotrophic keratitis, an uncommon degenerative disease of the cornea resulting in denervation, breakdown of the epithelium, ulceration and perforation, (59, 60), recombinant nerve growth factor (cenegermin) stimulated the reinnervation of rabbit corneas after refractive surgery (61) and boosted corneal wound healing in human patients. However, in controlled clinical studies it has failed to show significant benefit over vehicle (artificial tears) in terms of corneal sensitivity or visual acuity (62). Moreover, its direct contribution to the regeneration and reinnervation of corneas from patients with neurotrophic keratitis and whether it can resolve dry eye pathologies in the setting of chronic inflammation are yet to be addressed.

Our study highlights a novel and significant link between neuroregeneration and the restoration of progenitor cell identity in a model of chronic, autoimmune-mediated dry eye. Multiple studies in other organ systems, such as the salivary glands (63), tooth (64) and skin (65, 66), have established a critical role for nerves in the regulation of tissue structure, and function, as well as progenitor cell maintenance. In the cornea, mouse models of both chemically- and physically-induced denervation (67, 68) have implicated sensory innervation as a regulator of progenitor cell identity, as determined by a reduction in the expression of epithelial progenitor cell markers such as p63 (67), an outcome that can be partially reversed by topical NGF treatment (68). Yet, the impact of denervation and reinnervation on the differentiation status of basal progenitors has not been addressed. Our data suggest that lacripep's restorative effects on corneal innervation are mediated through its impact on epithelial cell identity/differentiation. Further studies are needed to identify the specific signaling systems activated by lacripep and the mechanism whereby sensory innervation regulates epithelial progenitor identity and cell fate.

Although the precise mechanism by which lacripep stimulates corneal reinnervation remains unclear, its known interaction with SDC1 suggests that lacripep mediates its neuroregenerative effects via binding and activating SDC1. Indeed, the nerve phenotype of the Aire KO cornea during dry eye disease progression strongly resembles that of the injured Sdc1-knockout cornea (34), as shown by the substantial reduction in intraepithelial nerve terminals and reinnervation. However, little is known about the downstream pathways that mediate neuroregeneration in response to SDC1. Our RNAseq analysis of the Aire KO cornea provides new insight into the potential mechanism of corneal reinnervation through the identification of 3 master regulators of the highly enriched neuronal gene sets, namely Isl1, Rest, and Rreb1, potentially directing lacripep-induced nerve remodeling, neurite outgrowth, and synapse formation and function. Specifically, these factors have been reported to orchestrate processes such as neurogenesis, synaptogenesis and/or axon regeneration (38-43)). Additionally, REST acts as a critical factor linking neuronal activity to the activation of intrinsic homeostasis and restoring physiological levels of activity throughout the entire neuronal network (69). Furthermore, in addition to regulating innervation, ISL1, RREB1 and REST are also involved in stem cell/progenitor maintenance, epithelial differentiation and proliferation, as well as epithelial architecture and integrity (70-74), suggesting lacripep delivers regenerative instructions to the epithelium to coordinate the regulation of tissue structure and cell identity. Thus, lacripep may act by fine-tuning the nerves and the epithelium via these TFs to achieve functional reinnervation.

In summary, our study highlights lacripep as a new therapeutic capable of resolving ocular damage through promoting functional reinnervation of the cornea. Future studies exploring lacripep's effects at the cellular level will allow us to identify and target specific signaling pathways essential for corneal re-innervation and restoration in patients with dry eye and other vision-threatening ocular surface disorders that impact corneal nerves (e.g., herpes simplex virus, interstitial keratitis, and neurotrophic keratitis).

### Materials and Methods

### Animal Model

All procedures were approved by the UCSF Institutional Animal Care and Use Committee (IACUC) and adhered to the NIH Guide for the Care and Use of Laboratory Animals (Approval number: 332AN089075-02). Wild type (WT) and Aire-deficient mice on the BALB/c background (BALB/c Aire KO) were the gift of Mark Anderson, University of California, San Francisco. Adult female mice (aged between 5 and 7 weeks) were used in all experiments. Mice were housed in the University of California, San Francisco Parnassus campus Laboratory Animal Resource Center (LARC), which is AAALAC accredited. Mice were housed in groups of up to five per cage where possible, in individually ventilated cages (IVCs), with fresh water, regular cleaning, and environmental enrichment. Appropriate sample size was calculated using power calculations. Genomic DNA isolated from tail clippings was genotyped for the Aire mutations by PCR with the recommended specific primers and their optimized PCR protocols (Jackson Laboratories Protocol 17936).

### Treatment Regimen

Aire KO female mice aged 5 weeks were used for the study. All mice were topically treated with 5 μL per eye of 4 μM lacripep. Dosing was three times daily for 14 consecutive days. Lissamine green staining assessment and tear secretion assays were performed on each eye before treatment at baseline, 7 days and 14 days post treatment

### Analysis of Epithelial Barrier Function

After mice were anesthetized with isoflurane, 5 μL of lissamine green dye (1%) was applied to the lower conjunctival cul-de-sac. Images of the cornea were then taken using an Olympus Zoom Stereo Microscope (Olympus, CenterValley, PA). Lissamine green staining was scored by dividing the cornea into four quadrants, the extent of staining in each quadrant was classified as Grade 0, no staining; Grade 1, sporadic (<25%); Grade 2, diffuse punctate (25-75%), or Grade 3, coalesced punctate staining (75% or more). The total score was calculated separately for each eye and equaled the sum of all four quadrants ranging from 0 (no staining) to 12 (most severe staining). Scoring was conducted by three masked observers with each data point representing the fold change of each eye relative to its baseline score before treatment.

### Tear Secretion Measurements

Mice were anesthetized with isoflurane and basal tear secretion was then measured using a Zone-Quick phenol red thread (as indicated by the length of the tear-absorbed region in 15 seconds). Stimulated tear secretion was measured after 4.5 mg/kg of pilocarpine diluted in saline was injected into the peritoneum (i.p.). Ten minutes later, mice were anesthetized with isoflurane and tear secretion was measured using a Zone-Quick phenol red thread (Showa Yakuhin Kako Co. Ltd., Tokyo, Japan).

### Tissue Processing and Immunohistological Analyses

Immunohistological and immunofluorescent analyses of cornea and lacrimal gland samples were performed as previously described (8). Briefly, enucleated eyes were embedded in OCT Tissue Tek freezing media. 7 μm and 20 μm sections were prepared from fresh frozen tissues using a cryostat (Leica, Izar, Germany) and mounted on SuperFrost Plus slides. Sections were fixed for 20 min in 4% paraformaldehyde (PFA) at room temperature (RT) and permeabilized using 0.3% Triton X100 in phosphate buffered saline for 15 min. Sections were then washed in PBS-Tween 20 (PBST) for 10 min, before being blocked with 5% normal donkey serum (Jackson Laboratories, ME) in PBST for 1 hour at RT. After blocking, slides were incubated with primary antibodies diluted in blocking buffer overnight at 4° C. Following 3 washes with PBST, slides were incubated with secondary antibodies diluted in blocking solution at RT for 1 hour.

For immunofluorescent analysis, 7 or 20 μm tissue sections were incubated with the following primary antibodies: mouse anti-ZO1 conjugated to Alexa594 (1:1000, Life Technologies, Cat 339194), rabbit anti-KRT6A (1:800, Cell Signaling, Cat 4912S), chicken anti-KRT14 (1:1500, Santa Cruz, Cat 515882), rat anti-Ki67 (1:200, Biolegend, 652405), rat anti-CD4 (1:200, Santa Cruz), rabbit anti-TUBB3 (1:500, Cell Signaling, Cat 5568S), and rat anti-Ecadherin (1:300, Life Technologies, Cat 131900). Antibodies were detected using Cy2-, Cy3- or Cy5-conjugated secondary Fab fragment antibodies (Jackson Laboratories), and nuclei were stained with Hoechst 33342 (1:3000, Sigma-Aldrich). Fluorescence was analyzed using a Zeiss LSM 900 confocal microscope or Zeiss Yokogawa Spinning disk confocal microscope with images assessed using NIH ImageJ software, as described below.

For corneal whole mount staining, PFA fixed corneas were blocked and incubated in primary antibodies for 48 hr at 4° C.: Mouse anti-TUBB3 (1:300, R&D, MAB 1195), rabbit anti-GAP43 (1:400, Novus bio, NB 300-143), goat anti-CGRP (1:300, Thermo fisher, PA1-85250), and rat anti-Substance P (1:500, Millipore, MAB 356). This was followed by extensive washes in PBST before tissue was incubated in secondary antibodies overnight at 4° C.

### In Situ Hybridization

Manual chromogenic RNAscope (ACDBio) was performed using company protocols on fresh frozen cornea tissue sections to detect target RNA at single cell level. Tissue pre-treatment included fixation for 15 min in 4% paraformaldehyde (PFA) at 4° C., RNAscope® Hydrogen Peroxide (ACD #322335) treatment for 10 min at RT followed by protease treatment (RNAscope® Protease Plus ACD #322331) for 10 min at 40° C. using the HybEZ Oven. Detection of specific probe binding sites was with RNAscope Reagent kit—from ACD (Cat. 323110). Single ISH detection for mouse Sdc1 (ACD Probe: 813921), Mouse Positive Control Probe (ACD Probe: 320881) and Negative Control Probe (ACD Probe: 320871) was performed manually. Target probes were hybridized for 2 hr at 40° C. using HybEZ oven followed by amplification steps according to the manufacture's protocol. Positive staining was indicated by fluorescent dots in the cell cytoplasm or nucleus.

### Image Analysis

Quantification of tight junction protein ZO1. ZO-1 fluorescent intensity was quantified in the apical squamous epithelial layer within a region of interest (ROI) containing a 350 μm section of central cornea epithelium. A Tsai's thresholding method (75) (Moments) was then applied to the ROI, and integrated densities within the ROI of the thresholded image were recorded.

Quantification of corneal epithelial basal progenitor identity and proliferation. The number of basal KRT14+ and KRT14+ KRT6A+ basal epithelial cells were counted in 350 μm sections of central cornea. To obtain the percentage of KRT6A+ basal cells per respective region, the number of KRT14+ KRT6A+ basal cells was divided by the total number of KRT14+ basal cells. The number of proliferating epithelial progenitors (Ki67+ KRT14+) was presented as a percentage of total KRT14+ cells counted in 350 μm sections of central cornea.

Quantification of CD4+ T cells in cornea and Lacrimal gland. The numbers of CD4+ T cells in the limbal stroma were counted and graphed. CD4+ T cells in the lacrimal gland were presented as percent coverage of total CD4+ T cells within the whole lacrimal gland tissue area.

Quantification of corneal epithelial nerve density and synapses. Tissue sections: TUBB3+ nerve density within 300 μm sections of central cornea epithelial ROI was quantified by applying Tsai's thresholding method (Moments), with integrated densities within the ROI of the thresholded image being recorded. Corneal whole mounts: The density of nerves expressing GAP43, TUBB3, or GAP43 and TUBB3 (% Area of 354 μm×396 μm) was quantified from central cornea images of whole mount cornea and plotted in a stacked bar graph to visualize the proportion of each of the three nerve types. Similarly, numbers of synapses marked by expression of GAP43, TUBB3, or GAP43 and TUBB3 were counted per respective region of central cornea (100 μm2) and plotted in a stacked bar graph. The density of GAP43+ nerves were quantified from the whole mount cornea images (354 μm×396 μm) applying Tsai's threshold as described above.

3D reconstruction of nerve fibers. Acquired confocal Z-stacks comprising epithelial and stroma were reconstructed into 3D images using Imaris image analysis software (Bitplane AG, Zurich, Switzerland). The surface of the nuclei (Hoechst) and nerves (TUBB3/GAP43/SP/CGRP) were segmented using the background subtraction threshold. Each channel was obtained separately. Touching nuclei were split by the calculated seed points inside each area.

### RNA Isolation and RNAseq Library Generation

Total RNA was collected at 7 days of treatment and purified using RNAaqueous and DNase reagents according to the manufacturer's instructions (Ambion, Houston, TX, USA). RNA quality was assessed using the Agilent 2100 BioAnalyzer, and samples with an RNA integrity ≥6.0 were included for RNA sequencing. The synthesis of mRNA libraries was performed by Novogene Corporation Inc. according to their protocols. RNA library was formed by ploy A capture (or rRNA removal), RNA fragmentation by covaris or enzyme digestion and reverse transcription of cDNA. Sequencing was performed as described below.

### RNAseq Analysis

RNA libraries were sequenced on an Illumina NovaSeq 6000. Depths of 20-30 million 150 bp paired-end reads were generated for each sample. Quality control metrics were performed on raw sequencing reads using the FASTQC v0.11.9 application (76). Reads were mapped to the UCSC Mus musculus genome mm 10 (NCBI build v38) using Spliced Transcripts Alignment to a Reference (STAR) (77). At least 90% of the reads were successfully mapped. Reads aligning to the mm 10 build were quantified against Ensembl Transcripts release 93 using Partek® E/M (Partek's optimization of the expectation maximization algorithm, Partek Inc, St. Louis, MO, USA), which disregarded any reads that aligned to more than one location or more than one gene at a single location. Data was normalized by two procedures: 1. total count normalization, 2. addition of a small offset (0.0001). DEseq2 was then used to detect differential gene expression between WT, untreated Aire KO, PBS treated Aire KO and lacripep treated Aire KO corneas based on the normalized count data. Genes were considered differentially expressed if the log 2 Fold Change between samples was at least 1, with the adjusted p-value held to 0.05 (36). Heatmaps and Volcano plot of differentially expressed genes were created using “pheatmap”, and “EnhancedVolcano” R packages, respectively (78, 79).

A list of all significantly modulated genes (p<0.05) was used as input for gene ontology (GO) analysis using the online Database for Annotation, Visualization and Integrated Discovery (DAVID, 2021 update (80). We consider an attribute to be significant if its adjusted p value is less than 0.05 relative to an appropriate background gene set.

To identify regulatory networks underlying the impact of lacripep on tissue regeneration, we used the cytoscape application iRegulon (37) to predict master regulators, i.e., transcription factors whose regulons (transcriptional target sets) are highly enriched with the input gene list. A list of all significantly modulated genes (FC>1.5, p<0.05) was used as input and the normalized enrichment score (NES) threshold was set to 3, which corresponds to an FDR of the TF recovery between 3%-9%. Exclusively upregulated genes for each TF were further used as input for GO analysis using DAVID as indicated above.

### Statistical Analysis

A minimum of three independent repeats were conducted in all experiments. Bar graphs are used to summarize the means and standard deviations of each outcome obtained using all data collected from wild type (WT) and Aire KO mice. A Student's t-test was used for two group comparisons; a one-way analysis of variance (ANOVA) was used for multiple group comparisons with either Tukey's multiple comparisons test, Dunnett T3, or corrected for multiple comparisons by controlling the False Discovery Rate using Benjamini, Krieger and Yekutieli; P<0.05 was considered statistically significant. A false discovery rate of 0.05 was applied to RNAseq data. A Wald test with Benjamini and Hochberg correction was used for differential gene expression, and a Fisher's exact test was used for Gene Ontology analysis.

### REFERENCES

1. M. A. Lemp, C. Baudouin, J. Baum, M. Dogru, G. N. Foulks, S. Kinoshita, P. Laibson, J. McCulley, J. Murube, S. C. Pflugfelder, M. Rolando, I. Toda, in Ocular Surface (2007).
2. S. C. Pflugfelder, C. S. de Paiva, The Pathophysiology of Dry Eye Disease: What We Know and Future Directions for Research. Ophthalmology. 124, S4-S13 (2017).
3. F. Stapleton, M. Alves, V. Y. Bunya, I. Jalbert, K. Lekhanont, F. Malet, K.-S. Na, D. Schaumberg, M. Uchino, J. Vehof, E. Viso, S. Vitale, L. Jones, TFOS DEWS II Epidemiology Report. Ocul. Surf. 15, 334-365 (2017).
4. T. Vijmasi, F. Y. T. Chen, S. Balasubbu, M. Gallup, R. L. Mckown, G. W. Laurie, N. A. McNamara, Topical Administration of Lacritin Is a Novel Therapy for Aqueous-Deficient Dry Eye Disease. Invest. Ophthalmol. Vis. Sci. 55, 5401-5409 (2014).
5. S. Samudre, F. A. Lattanzio Jr, V. Lossen, A. Hosseini, J. D. Sheppard Jr, R. L. Mckown, G. W. Laurie, P. B. Williams, Lacritin, a novel human tear glycoprotein, promotes sustained basal tearing and is well tolerated. Invest. Ophthalmol. Vis. Sci. 52, 6265-6270 (2011).
6. J. Wang, N. Wang, J. Xie, S. C. Walton, R. L. Mckown, R. W. Raab, P. Ma, S. L. Beck, G. L. Coffman, I. M. Hussaini, G. W. Laurie, Restricted epithelial proliferation by lacritin via PKCα-dependent NFAT and mTOR pathways. J. Cell Biol. 174, 689-700 (2006).
7. G. A. Georgiev, M. S. Gh., J. Romano, K. L. Dias Teixeira, C. Struble, D. S. Ryan, R. K. Sia, J. P. Kitt, J. M. Harris, K.-L. Hsu, A. Libby, M. G. Odrich, T. Suárez, R. L. Mckown, G. W. Laurie, Lacritin proteoforms prevent tear film collapse and maintain epithelial homeostasis. J. Biol. Chem. 296, 100070 (2021).
8. Y. Efraim, F. Y. T. Chen, C. Stashko, K. N. Cheong, E. Gaylord, N. McNamara, S. M. Knox, Alterations in corneal biomechanics underlie early stages of autoimmune-mediated dry eye disease. J. Autoimmun. 114, 102500 (2020).
9. F. Y. Chen, A. Lee, S. Ge, S. Nathan, S. M. Knox, N. A. McNamara, Aire-deficient mice provide a model of corneal and lacrimal gland neuropathy in Sjogren's syndrome. PLOS One (2017), doi: 10.1371/journal.pone.0184916.
10. F. Y. T. Chen, E. Gaylord, N. McNamara, S. Knox, Deciphering molecular and phenotypic changes associated with early autoimmune disease in the aire-deficient mouse model of Sjögren's syndrome. Int. J. Mol. Sci. (2018), doi: 10.3390/ijms19113628.
11. F. Mantelli, J. Mauris, P. Argüeso, The ocular surface epithelial barrier and other mechanisms of mucosal protection: from allergy to infectious diseases. Curr. Opin. Allergy Clin. Immunol. (2013), doi: 10.1097/ACI.0b013e3283645899
12. S. P. Sugrue, J. D. Zieske, ZO1 in corneal epithelium: Association to the zonula occludens and adherens junctions. Exp. Eye Res. (1997), doi: 10.1006/exer.1996.0175.
13. N. Di Girolamo, S. Bobba, V. Raviraj, N. C. Delic, I. Slapetova, P. R. Nicovich, G. M. Halliday, D. Wakefield, R. Whan, J. G. Lyons, Tracing the fate of limbal epithelial progenitor cells in the murine cornea. Stem Cells (2015), doi: 10.1002/stem. 1769.
14. M. Park, A. Richardson, E. Pandzic, E. P. Lobo, R. Whan, S. L. Watson, J. G. Lyons, D. Wakefield, N. Di Girolamo, Visualizing the contribution of keratin-14+ limbal epithelial precursors in corneal wound healing. Stem cell reports. 12, 14-28 (2019).
15. K. E. Boonekamp, K. Kretzschmar, D. J. Wiener, P. Asra, S. Derakhshan, J. Puschhof, C. López-Iglesias, P. J. Peters, O. Basak, H. Clevers, Long-term expansion and differentiation of adult murine epidermal stem cells in 3D organoid cultures. Proc. Natl. Acad. Sci. U.S.A. 116, 14630-14638 (2019).
16. A. Schermer, J. V Jester, C. Hardy, D. Milano, T.-T. Sun, Transient synthesis of K6 and K16 keratins in regenerating rabbit corneal epithelium: keratin markers for an alternative pathway of keratinocyte differentiation. Differentiation. 42, 103-110 (1989).
17. S. C. Pflugfelder, C. S. de Paiva, D.-Q. Li, M. E. Stern, Epithelial-immune cell interaction in dry eye. Cornea. 27, S9 (2008).
18. D. Zhou, Y.-T. Chen, F. Chen, M. Gallup, T. Vijmasi, A. F. Bahrami, L. B. Noble, N. van Rooijen, N. A. McNamara, Critical Involvement of Macrophage Infiltration in the Development of Sjögren's Syndrome-Associated Dry Eye. Am. J. Pathol. 181, 753-760 (2012).
19. Y. Ohno, K. Satoh, A. Shitara, T. Into, M. Kashimata, Arginase 1 is involved in lacrimal hyposecretion in male NOD mice, a model of Sjögren's syndrome, regardless of dacryoadenitis status. J. Physiol. 598, 4907-4925 (2020).
20. A. Labbé, Q. Liang, Z. Wang, Y. Zhang, L. Xu, C. Baudouin, X. Sun, Corneal Nerve Structure and Function in Patients With Non-Sjögren Dry Eye: Clinical Correlations. Invest. Ophthalmol. Vis. Sci. 54, 5144-5150 (2013).
21. F. Li, Q. Zhang, X. Ying, J. He, Y. Jin, H. Xu, Y. Cheng, M. Zhao, Corneal nerve structure in patients with primary Sjögren's syndrome in China. BMC Ophthalmol. 21, 211 (2021).
22. C. Simsek, T. Kojima, M. Dogru, K. Tsubota, Alterations of Murine Subbasal Corneal Nerves After Environmental Dry Eye Stress. Invest. Ophthalmol. Vis. Sci. 59, 1986-1995 (2018).
23. R. Navone, C. Lunardi, R. Gerli, E. Tinazzi, D. Peterlana, C. Bason, R. Corrocher, A. Puccetti, Identification of tear lipocalin as a novel autoantigen target in Sjögren's syndrome. J. Autoimmun. (2005), doi: 10.1016/j.jaut.2005.09.021.
24. N. A. McNamara, S. Ge, S. M. Lee, A. M. Enghauser, L. Kuehl, F. Y.-T. Chen, M. Gallup, R. L. Mckown, Reduced Levels of Tear Lacritin Are Associated With Corneal Neuropathy in Patients With the Ocular Component of Sjögren's Syndrome. Invest. Ophthalmol. Vis. Sci. 57, 5237-5243 (2016).
25. R. E. Martin, N. G. Bazan, Growth-associated protein GAP-43 and nerve cell adhesion molecule in sensory nerves of cornea. Exp. Eye Res. 55, 307-314 (1992).
26. I. Kovács, A. Ludány, T. Köszegi, J. Fehér, B. Kovács, J. Szolcsányi, E. Pintér, Substance P released from sensory nerve endings influences tear secretion and goblet cell function in the rat. Neuropeptides. 39, 395-402 (2005).
27. H. Uusitalo, K. Krootila, A. Palkama, Calcitonin gene-related peptide (CGRP) immunoreactive sensory nerves in the human and guinea pig uvea and cornea. Exp. Eye Res. 48, 467-475 (1989).
28. A. A. MIKULEC, D. L. TANELIAN, CGRP increases the rate of corneal re-epithelialization in an in vitro whole mount preparation. J. Ocul. Pharmacol. Ther. 12, 417-423 (1996).
29. G. Quadrato, S. Di Giovanni, Waking up the sleepers: shared transcriptional pathways in axonal regeneration and neurogenesis. Cell. Mol. Life Sci. 70, 993-1007 (2013).
30. A. Tedeschi, F. Bradke, Spatial and temporal arrangement of neuronal intrinsic and extrinsic mechanisms controlling axon regeneration. Curr. Opin. Neurobiol. 42, 118-127 (2017).
31. M. Mahar, V. Cavalli, Intrinsic mechanisms of neuronal axon regeneration. Nat. Rev. Neurosci. 19, 323-337 (2018).
32. P. Ma, S. L. Beck, R. W. Raab, R. L. Mckown, G. L. Coffman, A. Utani, W. J. Chirico, A. C. Rapraeger, G. W. Laurie, Heparanase deglycanation of syndecan-1 is required for binding of the epithelial-restricted prosecretory mitogen lacritin. J. Cell Biol. 174, 1097-1106 (2006)
33. C. Lee, M. C. Edman, G. W. Laurie, S. F. Hamm-Alvarez, J. A. MacKay, Biosynthesized Multivalent Lacritin Peptides Stimulate Exosome Production in Human Corneal Epithelium. Int. J. Mol. Sci . . . 21 (2020), doi: 10.3390/ijms21176157.
34. S. Pal-Ghosh, G. Tadvalkar, M. A. Stepp, Alterations in Corneal Sensory Nerves During Homeostasis, Aging, and After Injury in Mice Lacking the Heparan Sulfate Proteoglycan Syndecan-1. Invest. Ophthalmol. Vis. Sci. 58, 4959-4975 (2017).
35. M. A. Stepp, H. E. Gibson, P. H. Gala, D. D. Drina, A. Pajoohesh-Ganji, S. Pal-Ghosh, M. Brown, C. Aquino, A. M. Schwartz, O. Goldberger, M. T. Hinkes, M. Bernfield, Defects in keratinocyte activation during wound healing in the syndecan-1-deficient mouse. J. Cell Sci. (2002), doi: 10.1242/jcs.00128.
36. M. I. Love, W. Huber, S. Anders, Moderated estimation of fold change and dispersion for RNA-seq data with DESeq2. Genome Biol. (2014), doi: 10.1186/s13059-014-0550-8.
37. R. Janky, A. Verfaillie, H. Imrichová, B. Van de Sande, L. Standaert, V. Christiaens, G. Hulselmans, K. Herten, M. Naval Sanchez, D. Potier, iRegulon: from a gene list to a gene regulatory network using large motif and track collections. PLOS Comput. Biol. 10, e1003731 (2014).
38. Q. Zhang, R. Huang, Y. Ye, X. Guo, J. Lu, F. Zhu, X. Gong, Q. Zhang, J. Yan, L. Luo, S. Zhuang, Y. Chen, X. Zhao, S. M. Evans, C. Jiang, X. Liang, Y. Sun, Temporal requirements for ISL1 in sympathetic neuron proliferation, differentiation, and diversification. Cell Death Dis. 9, 247 (2018).
39. Y. Sun, I. M. Dykes, X. Liang, S. R. Eng, S. M. Evans, E. E. Turner, A central role for Islet1 in sensory neuron development linking sensory and spinal gene regulatory programs. Nat. Neurosci. 11, 1283-1293 (2008).
40. N. Ballas, G. Mandel, The many faces of REST oversee epigenetic programming of neuronal genes. Curr. Opin. Neurobiol. 15, 500-506 (2005)
41. J.-Y. Hwang, R. S. Zukin, REST, a master transcriptional regulator in neurodegenerative disease. Curr. Opin. Neurobiol. 48, 193-200 (2018).
42. A. Rodenas-Ruano, A. E. Chávez, M. J. Cossio, P. E. Castillo, R. S. Zukin, REST-dependent epigenetic remodeling promotes the developmental switch in synaptic NMDA receptors. Nat. Neurosci. 15, 1382-1390 (2012).
43. J. E. Farley, T. C. Burdett, R. Barria, L. J. Neukomm, K. P. Kenna, J. E. Landers, M. R. Freeman, Transcription factor Pebbled/RREB1 regulates injury-induced axon degeneration. Proc. Natl. Acad. Sci. 115, 1358-1363 (2018).
44. T. Nieto-Miguel, M. Calonge, A. de la Mata, M. López-Paniagua, S. Galindo, M. F. de la Paz, R. M. Corrales, A comparison of stem cell-related gene expression in the progenitor-rich limbal epithelium and the differentiating central corneal epithelium. Mol. Vis. 17, 2102-2117 (2011).
45. N. Bouheraoua, S. Fouquet, M. T. Marcos-Almaraz, D. Karagogeos, L. Laroche, A. Chédotal, Genetic Analysis of the Organization, Development, and Plasticity of Corneal Innervation in Mice. J. Neurosci. 39, 1150-1168 (2019).
46. M. C. Mocan, I. Durukan, M. Irkec, M. Orhan, Morphologic alterations of both the stromal and subbasal nerves in the corneas of patients with diabetes. Cornea. 25, 769-773 (2006).
47. E. Villani, D. Galimberti, F. Viola, C. Mapelli, N. Del Papa, R. Ratiglia, Corneal involvement in rheumatoid arthritis: an in vivo confocal study. Invest. Ophthalmol. Vis. Sci. 49, 560-564 (2008).
48. E. Szalai, G. Szucs, S. Szamosi, Z. Aszalos, I. Afra, A. Kemeny-Beke, An in vivo confocal microscopy study of corneal changes in patients with systemic sclerosis. Sci. Rep. 11, 1-7 (2021).
49. L.-Q. Wu, P. Mou, Z.-Y. Chen, J.-W. Cheng, Q.-H. Le, J.-P. Cai, R.-L. Wei, Altered corneal nerves in Chinese thyroid-associated ophthalmopathy patients observed by in vivo confocal microscopy. Med. Sci. Monit. Int. Med. J. Exp. Clin. Res. 25, 1024 (2019).
50. B. Steger, L. Speicher, W. Philipp, N. E. Bechrakis, In vivo confocal microscopic characterisation of the cornea in chronic graft-versus-host disease related severe dry eye disease. Br. J. Ophthalmol. 99, 160-165 (2015).
51. A. Altshuler, A. Amitai-Lange, N. Tarazi, S. Dey, L. Strinkovsky, S. Hadad-Porat, S. Bhattacharya, W. Nasser, J. Imeri, G. Ben-David, Discrete limbal epithelial stem cell populations mediate corneal homeostasis and wound healing. Cell Stem Cell. 28, 1248-1261 (2021).
52. J. Liu, Z. Li, Resident innate immune cells in the cornea. Front. Immunol. 12, 216 (2021).
53. A. Namavari, S. Chaudhary, J.-H. Chang, L. Yco, S. Sonawane, V. Khanolkar, B. Y. Yue, J. Sarkar, S. Jain, Cyclosporine immunomodulation retards regeneration of surgically transected corneal nerves. Invest. Ophthalmol. Vis. Sci. 53, 732-740 (2012).
54. I. Alcalde, A. Inigo-Portugues, N. Carreno, A. C. Riestra, J. M. Merayo-Lloves, Effects of new biomimetic regenerating agents on corneal wound healing in an experimental model of post-surgical corneal ulcers. Arch. la Soc. Española Oftalmol. (English Ed. 90, 467-474 (2015).
55. C. Wang, Y. Peng, S. Pan, L. Li, Effect of insulin-like growth factor-1 on corneal surface ultrastructure and nerve regeneration of rabbit eyes after laser in situ keratomileusis. Neurosci. Lett. 558, 169-174 (2014).
56. C. Fukiage, T. Nakajima, Y. Takayama, Y. Minagawa, T. R. Shearer, M. Azuma, PACAP induces neurite outgrowth in cultured trigeminal ganglion cells and recovery of corneal sensitivity after flap surgery in rabbits. Am. J. Ophthalmol. 143, 255-262 (2007).
57. M. S. Cortina, J. He, N. Li, N. G. Bazan, H. E. P. Bazan, Recovery of corneal sensitivity, calcitonin gene-related peptide-positive nerves, and increased wound healing induced by pigment epithelial-derived factor plus docosahexaenoic acid after experimental surgery. Arch. Ophthalmol. 130, 76-83 (2012).
58. C. Yabuta, T. Oka, Y. Kishimoto, A. Ohtori, A. Yoshimatsu, M. Azuma, Topical FK962 facilitates axonal regeneration and recovery of corneal sensitivity after flap surgery in rabbits. Am. J. Ophthalmol. 153, 651-660 (2012).
59. S. C. Pflugfelder, M. Massaro-Giordano, V. L. Perez, P. Hamrah, S. X. Deng, L. Espandar, C. S. Foster, J. Affeldt, J. A. Seedor, N. A. Afshari, Topical recombinant human nerve growth factor (cenegermin) for neurotrophic keratopathy: a multicenter randomized vehicle-controlled pivotal trial. Ophthalmology. 127, 14-26 (2020).
60. S. Bonini, A. Lambiase, P. Rama, F. Sinigaglia, M. Allegretti, W. Chao, F. Mantelli, E. Messmer, P. Aragona, G. Geerling, Phase II randomized, double-masked, vehicle-controlled trial of recombinant human nerve growth factor for neurotrophic keratitis. Ophthalmology. 125, 1332-1343 (2018).
61. Q. Gong, S. Zhang, L. Jiang, M. Lin, Z. Xu, Y. Yu, Q. Wang, F. Lu, L. Hu, The effect of nerve growth factor on corneal nerve regeneration and dry eye after LASIK. Exp. Eye Res. 203, 108428 (2021).
62. E. D. Deeks, Y. N. Lamb, Cenegermin: a review in neurotrophic keratitis. Drugs. 80, 489-494 (2020).
63. E. Emmerson, A. J. May, L. Berthoin, N. Cruz-Pacheco, S. Nathan, A. J. Mattingly, J. L. Chang, W. R. Ryan, A. D. Tward, S. M. Knox, Salivary glands regenerate after radiation injury through SOX2-mediated secretory cell replacement. EMBO Mol. Med. 10, e8051 (2018).
64. H. Zhao, J. Feng, K. Seidel, S. Shi, O. Klein, P. Sharpe, Y. Chai, Secretion of shh by a neurovascular bundle niche supports mesenchymal stem cell homeostasis in the adult mouse incisor. Cell Stem Cell. 14, 160-173 (2014).
65. X. Ying, T. D. T., W. J. S., W. Chaochen, P. James, P. Ralitsa, B. Isaac, Neural Hedgehog signaling maintains stem cell renewal in the sensory touch dome epithelium. Proc. Natl. Acad. Sci. 112, 7195-7200 (2015).
66. S. Huang, P. Kuri, Y. Aubert, M. Brewster, N. Li, O. Farrelly, G. Rice, H. Bae, S. Prouty, T. Dentchev, W. Luo, B. C. Capell, P. Rompolas, Lgr6 marks epidermal stem cells with a nerve-dependent role in wound re-epithelialization. Cell Stem Cell. 28, 1582-1596.e6 (2021).
67. H. Ueno, G. Ferrari, T. Hattori, D. R. Saban, K. R. Katikireddy, S. K. Chauhan, R. Dana, Dependence of corneal stem/progenitor cells on ocular surface innervation. Invest. Ophthalmol. Vis. Sci. 53, 867-872 (2012).
68. A. Lambiase, L. Aloe, F. Mantelli, M. Sacchetti, E. Perrella, P. Bianchi, M. L. Rocco, S. Bonini, Capsaicin-Induced Corneal Sensory Denervation and Healing Impairment Are Reversed by NGF Treatment. Invest. Ophthalmol. Vis. Sci. 53, 8280-8287 (2012).
69. D. Pozzi, G. Lignani, E. Ferrea, A. Contestabile, F. Paonessa, R. D'Alessandro, P. Lippiello, D. Boido, A. Fassio, J. Meldolesi, F. Valtorta, F. Benfenati, P. Baldelli, REST/NRSF-mediated intrinsic homeostasis protects neuronal networks from hyperexcitability. EMBO J. 32, 2994-3007 (2013).
70. B. L. Baechler, C. McKnight, P. C. Pruchnicki, N. A. Biro, B. H. Reed, Hindsight/RREB-1 functions in both the specification and differentiation of stem cells in the adult midgut of Drosophila. Biol. Open. 5, 1-10 (2015).
71. S. M. Morgani, J. Su, J. Nichols, J. Massagué, A.-K. Hadjantonakis, The transcription factor Rreb1 regulates epithelial architecture, invasiveness, and vasculogenesis in early mouse embryos. Elife. 10, e64811 (2021).
72. S. K. Singh, M. N. Kagalwala, J. Parker-Thornburg, H. Adams, S. Majumder, REST maintains self-renewal and pluripotency of embryonic stem cells. Nature. 453, 223-227 (2008).
73. T. Su, H. Liu, D. Zhang, G. Xu, J. Liu, S. M. Evans, J. Pan, S. Cui, LIM homeodomain transcription factor Isl1 affects urethral epithelium differentiation and apoptosis via Shh. Cell Death Dis. 10, 1-12 (2019).
74. E. Kim, M. Jiang, H. Huang, Y. Zhang, N. Tjota, X. Gao, J. Robert, N. Gilmore, L. Gan, J. Que, Isl1 regulation of Nkx2. 1 in the early foregut epithelium is required for trachea-esophageal separation and lung lobation. Dev. Cell. 51, 675-683 (2019).
75. W.-H. Tsai, Moment-preserving thresolding: A new approach. Comput. Vision, Graph. Image Process. 29, 377-393 (1985).
76. S. Andrews, FastQC: a quality control tool for high throughput sequence data (2010).
77. A. Dobin, C. A. Davis, F. Schlesinger, J. Drenkow, C. Zaleski, S. Jha, P. Batut, M. Chaisson, T. R. Gingeras, STAR: Ultrafast universal RNA-seq aligner. Bioinformatics (2013), doi: 10.1093/bioinformatics/bts635.
78. K. Blighe, EnhancedVolcano: Publication-ready volcano plots with enhanced colouring and labeling. R package version 1.2. 0 (2019).
79. R. Kolde, Pheatmap: pretty heatmaps. R Packag. version. 1, 747 (2012).
80. D. W. Huang, B. T. Sherman, R. A. Lempicki, Systematic and integrative analysis of large gene lists using DAVID bioinformatics resources. Nat. Protoc. (2009), doi: 10.1038/nprot.2008.211.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of regenerating functional sensory nerves in an eye of a human having damaged corneal nerves, the method comprising,
contacting the eye of the human with a polypeptide comprising KQFIENGSEFAQKLLKKFSLLKPWA (SEQ ID NO:1) in a dosage sufficient to regenerate functional sensory nerves in the eye.

2. The method of claim 1, wherein the human has an eye disorder selected from the group consisting of neurotrophic keratitis, Sjogrens, rheumatoid arthritis, Crohn's disease, radiation-damage (keratopathy), diabetic neuropathy, keratoconus, infectious keratitis, herpes simplex, herpes zoster, corneal dystrophies, atopic keratoconjunctivis, allergic conjunctivitis, glaucoma, Stevens-Johnson syndrome, toxic epidermal necrolysis, limbal stem cell deficiency, corneal pain, corneal neuralgia, penetrating keratoplasty, phototherapeutic keratectomy, chemotherapy-induced peripheral neuropathies, neuropathic dry eye and Parkinson's disease.

3. The method of claim 1, wherein the human has an eye disorder resulting from laser epithelial keratomileusis (LASEK), laser-assisted in situ keratomileusis (LASIK), photorefractive keratectomy (PRK), or small incision lenticule extraction (SMILE).

4. The method of claim 1, wherein the eye tissue has damaged corneal architecture and dosage is sufficient to improve corneal architecture.

5. The method of claim 1, wherein the polypeptide has an amino acid sequence that consists of SEQ ID NO:1.

6. The method of claim 1, wherein the polypeptide consists of Ac-KQFIENGSEFAQKLLKKFSLLKPWA-NH₂ or a salt thereof.

7. The method of claim 1, wherein the dosage is administered under a contact lens.

8. The method of claim 1, wherein the polypeptide comprises or is linked to a protein domain that adheres to the surface of the eye.

9. The method of claim 8, wherein the protein domain binds to collagen, heparin, heparan sulfate, or a carbohydrate.

10. The method of claim 9, wherein the protein domain is selected from the group consisting of a lectin carbohydrate-binding anchor domain, a von Willebrand factor (vWF) collagen-binding anchor domain, a Clostridium collagenase (ColH) collagen-binding anchor domain, and a heparin-binding (HS) anchor domain.

11. A method of stimulating nerve regeneration in skin in a human in need thereof, the method comprising contacting the skin with a polypeptide comprising KQFIENGSEFAQKLLKKFSLLKPWA in an amount sufficient to stimulate nerve regeneration in the skin.

12. The method of claim 11, wherein the human has a peripheral neuropathy resulting from a disorder selected from the group consisting of Systemic Lupus Erythematosus, diabetic neuropathy, radiation exposure, traumatic injuries or toxic agents, and wherein at least one symptom of the disorder is ameliorated.

13. The method of claim 11, wherein the polypeptide consists of SEQ ID NO:1.

14. The method of claim 11, wherein the polypeptide has an amino acid sequence that consists of Ac-KQFIENGSEFAQKLLKKFSLLKPWA-NH₂ or a salt thereof.

15. A composition for ocular delivery comprising a polypeptide comprising KQFIENGSEFAQKLLKKFSLLKPWA (SEQ ID NO:1), wherein the polypeptide comprises or is linked to a protein domain that adheres to the surface of the eye.

16. The composition of claim 15, wherein the protein domain binds to collagen, heparin, heparan sulfate, or a carbohydrate.

17. The composition of claim 16, wherein the protein domain is selected from the group consisting of a lectin carbohydrate-binding anchor domain, a von Willebrand factor (vWF) collagen-binding anchor domain, a Clostridium collagenase (ColH) collagen-binding anchor domain, and a heparin-binding (HS) anchor domain.


SEQUENCE LISTING (5 sequences)

SEQ ID NO: 1 - protein, 25 aa, synthetic construct
  REGION 1..25: note = Description of sequence: component of regenerating peptide
KQFIENGSEFAQKLLKKFSLLKPWA

SEQ ID NO: 2 - protein, 138 aa, Homo sapiens
  REGION 1..138: note = Description of sequence: Lacritin
MKFTTLLFLAAVAGALVYAEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 3 - protein, 21 aa, synthetic construct
  REGION 1..21: note = Description of sequence: heparin-binding polypeptide
KRKKKGKGLGKKRDPSLRKYK

SEQ ID NO: 4 - protein, 21 aa, synthetic construct
  REGION 1..21: note = Description of sequence: heparin-binding polypeptide
KRKKKGKGLGKKRDPCLRKYK

SEQ ID NO: 5 - protein, 25 aa, synthetic construct
  REGION 1..25: note = Description of sequence: component of regenerating peptide
  MOD_RES <1: note = amino terminus is acetylated
  MOD_RES >25: note = carboxyl terminus is aminated
KQFIENGSEFAQKLLKKFSLLKPWA